(19) United States
(12) Patent Application Publication
(10) Pub. No.: US 2025/0360339 A1
(43) Pub. Date: Nov. 27, 2025

(54) MARKERLESS ANATOMICAL OBJECT TRACKING DURING AN IMAGE-GUIDED MEDICAL PROCEDURE

(71) Applicant: SEETREAT PTY LTD, Haymarket Sydney, New South Wales (AU)

(72) Inventors: Adam MYLONAS, Greenwich, New South Wales (AU); Marco MULLER, Greenwich, New South Wales (AU); Paul KEALL, Greenwich, New South Wales (AU); Jeremy BOOTH, Greenwich, New South Wales (AU); Doan Trang NGUYEN, Bexley North, New South Wales (AU)

(21) Appl. No.: 18/871,710

(22) PCT Filed: Jun. 6, 2023
(86) PCT No.: PCT/AU2023/050495

§ 371 Date: Dec. 4, 2024

Related U.S. Application Data
(60) Provisional application No. 63/349,486, filed on Jun. 6, 2022.

(51) Int. Cl. A61N 5/10; G06T 7/00; G06T 7/70

(52) U.S. Cl. CPC A61N 5/1049; G06T 7/0012; G06T 7/70; A61N 2005/1061; G06T 2207/10116; G06T 2207/20084; G06T 2207/30024

(57) ABSTRACT

An image guidance method for treatment by a medical device. The method comprises imaging a target area to which the treatment is to be delivered. During the interventional procedure, an image from the imaging is analysed by a patient-specific, individually trained artificial neural network to determine the position of at least one or more anatomical objects of interest present in the target area. The determined position(s) is output to the medical device for the delivery of treatment.

### FIELD OF THE INVENTION

The present invention relates generally to image guidance for a medical procedure, in particular, an interventional procedure such as guided radiation therapy, to treat a patient. Other interventional procedures are envisaged such as needle biopsy or minimally invasive surgery. In one form, there is disclosed a method and system for guiding a radiation therapy system by direct reference to the position of an anatomical object (e.g. soft tissue such as organs or tumours, or hard tissue like bone) to be radiated. The present invention does not require fiducial markers implanted in the target prior to treatment commencement.

### BACKGROUND OF THE INVENTION

Radiation therapy is a treatment modality used to treat localised tumours. It generally involves producing high energy megavoltage (MV) and conformal beams of X-rays to the target (tumour) using a medical linear accelerator. The radiation interacts with the tissues to create double strand DNA breaks to kill tumour cells. Radiation therapy requires high precision to deliver the dose to the tumour and spare healthy tissue, particularly that of organs surrounding the tumour. Each treatment is tailored to the individual patient.

Advances in radiation therapy techniques, such as intensity modulated radiation therapy (IMRT) and image guided radiation therapy (IGRT) have resulted in improved delivery of radiation doses to tumours while reducing normal tissue toxicity. According to current practices, IGRT is routinely applied at the start of treatment to align the target with its planned position. However, tumours in the head, heck, thorax, abdomen and pelvis are not static during treatment; a phenomenon known as ‘intrafraction motion’. Intrafraction motion occurs when patients move while on the treatment bed (both during setup and treatment) or when organs and tumours move in response to breathing and/or other voluntary movements or involuntary physiological processes such as bladder filling.

Regarding radiation therapy, the tumour and the surrounding anatomy are not static during the treatment. Therefore, image guidance during radiation therapy is required to monitor tumour motion and ensure adequate dose coverage of the target. Motion monitoring is essential for high dose treatments, such as stereotactic body radiation therapy (SBRT), where relatively high radiation dose per fraction is prescribed, with small geometric margins for treatment demanding high precision. For slow-moving tumours, the effect of intrafraction motion can result in up to 19% less radiation dose delivered to the target in one fraction compared to the prescribed dose per fraction. 13% of SBRT prostate cancer patients would not receive within 5% of the prescription without real-time motion adaptation. With mounting evidence on the detrimental effects of tumour motion during treatment, the American Society for Radiation Oncology recommended imaging during treatment to continuously monitor the tumour motion for high dose radiation treatments.

In the case of prostate cancer radiotherapy treatments, studies with electromagnetic transponders showed that the prostate can travel up to 15 mm during treatment (Langen et al. 2008). As prostate stereotactic body radiotherapy (SBRT) treatments become clinical standard, it is recommended that real-time motion monitoring is used during these high dose treatments to ensure the dose delivered faithfully reflects the treatment plan (Lovelock et al. 2014). A number of different intrafraction real-time guidance methods have been used during prostate cancer treatments. Systems such as CyberKnife (Accuray, Sunnyvale, CA) and the real-time tracking radiotherapy (RTRT) system use real-time kilovoltage (kV) images from two (CyberKnife) or four (RTRT system) orthogonal room-mounted imagers to track the prostate position based on segmented positions of implanted fiducial markers (King et al. 2009, Kitamura et al. 2002, Sazawa et al. 2009, Shimizu et al. 2000, Shirato et al. 2003, 2000). The commercial systems Calypso (Varian, Palo Alto, CA) (Kupelian et al. 2007) and RayPilot (Micropos, Gothenburg, Sweden) (Castellanos et al. 2012) utilise implanted electromagnetic transponders, transmitting positional signals to an external receiver. Emerging real-time guidance technologies include ultrasonography (Ballhausen et al. 2015) and integrated magnetic resonance imaging (MRI)-radiation therapy systems (Fallone et al. 2009, Raaymakers et al. 2009). Common to all these methods is the need for additional dedicated and typically expensive equipment as well as implantation of markers in the prostate for tracking to perform real-time guidance.

Real-time image guided adaptive radiation therapy (IGART) systems have been developed at least in part to account for this intrafraction motion. Real-time IGART can track the target and account for the motion. Typically, fiducial markers are implanted as a surrogate of the tumour position due to the low radiographic contrast of the soft tissues in kilovoltage (kV) X-ray images.

For the purposes of this invention, “real time” has its ordinary meaning of the actual time when a process or event occurs. This implies in computing that the input data is processed within milliseconds so that it is (or perceived as) available almost immediately as feedback.

Certain IGRT and IGART systems operate in real-time by utilising kilovoltage (kV) images for the tracking of fiducial markers implanted in tumours. One such system is known as Kilovoltage Intrafraction Monitoring (KIM). KIM is a real-time image guidance technique that utilises existing radiotherapy technologies found in cancer care centres (i.e. on-board X-ray images). KIM exploits fiducial markers implanted inside the tumour (organ) and reconstructs their location by acquiring multiple images of the target using the on-board kilovoltage (KV) beam (which is a low energy X-ray imager) and determining any motion in the left-right (LR), superior-inferior (SI), and anterior-posterior (AP) directions. KIM Tracking has also been developed, which dynamically modifies the position of a multi leaf collimator (MLC) while delivering the treatment dose based on the tumour position reconstructed by KIM. In KIM, tumour motion is monitored in real-time while both the MV beam is delivering the treatment dose, and the KV beam is imaging the tumour target. If significant motion away from the treatment beam occurs, the treatment is paused and the patient is repositioned before the treatment is continued.

Real-time IGART can track the target tumour during radiation therapy to improve target dose coverage and reduce the radiation dose to healthy tissue. IGART can be performed using kilovoltage (kV) projections from the X-ray imaging system on conventional radiation therapy treatment systems. A robust segmentation method of the target in each projection is required to accurately determine the target position. For conventional treatment systems, real-time motion monitoring methods typically track implanted fiducial markers as surrogates to the tumour, especially for organs and tumours with low radiographic contrast, such as the prostate. Fiducial markers and the insertion procedure results in added time delays, additional costs, and risks. The treatment delays are due to surgery wait time and the time for the markers to stabilise. The risks associated with the surgical implantation of markers include infection, haematuria, bleeding, and patient discomfort from the surgery. Furthermore, marker migration can result in tracking errors. Currently, patients who are not candidates for markers due to contraindications or are located in regional areas cannot receive real-time IGART.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

It is, accordingly, an object of the present invention to provide an alternative approach to segmentation for use in real-time systems without requiring implantation of fiducial markers (for example, gold seed fiducial markers to improve contrast in X-ray and CT images) in the tumour that is to be radiated.

### SUMMARY OF THE INVENTION

In one aspect, the invention provides an image guidance method for treatment by a medical device. The method comprises imaging a target area to which the treatment is to be delivered. The method also comprises during the interventional procedure, analysing an image from the imaging with a patient-specific, individually trained artificial neural network to determine the position of at least one or more anatomical objects of interest present in the target area. The method also comprises outputting the determined position(s).

The artificial neural network may be a conditional Generative Adversarial Network (cGAN).

The treatment may be an interventional procedure being any one from the group consisting of: guided radiation therapy, needle biopsy and minimally invasive surgery.

The anatomical object of interest may be any one from the group of: soft tissue and hard tissue.

The soft tissue may an organ or tumour.

The image may be an X-ray image.

The determined position(s) may be output to a radiation therapy system for the guided radiation therapy.

The method may further comprise: identifying the target area to which radiation is to be delivered on a basis of the outputted positions.

The method may further comprise: directing a treatment beam from the radiation therapy system based on a position of the identified target area.

The method may further comprise: tracking the target area by reference to successive output of positions over time; and directing the treatment beam at the target based on said tracking.

Directing the beam based on the position of the identified target area may include adjusting or setting one or more of the following parameters of the radiation therapy system: at least one geometrical property of said at least one emitted beam; a position of the target relative to the beam; a time of emission of the beam; and an angle of emission of the beam relative to the target area about a system rotational axis.

In a second aspect, there is provided an image guidance system for treatment provided by a medical device. The system comprises an imaging system arranged to generate a succession of images of a target area for directing the treatment provided by the medical device. The system also comprises a control system configured to: receive images from the imaging system; analyse the images with a patient-specific, individually trained artificial neural network during the treatment to: determine the position of the target area; and adjust the medical device using the determined positions to direct the treatment to the target area.

The artificial neural network may be a conditional Generative Adversarial Network (cGAN).

The treatment may be guided radiation therapy.

The medical device may be a radiation therapy treatment system comprising a radiation source for emitting at least one treatment beam of radiation.

The treatment beam may be directed to the target area.

In a third aspect, there is a computer software product comprising a sequence of instructions storable on one or more computer-readable storage media, said instructions when executed by one or more processors, cause the processor to: receive an image, from an imaging system, of a target area for directing treatment by a medical device; analyse the image with a patient-specific, individually trained artificial neural network to determine the position of one or more anatomical objects of interest present in the target area; and output the position of the one or more anatomical objects of interest to the medical device.

In a fourth aspect, there is provided a method of monitoring movement of an organ or portion of an organ or surrogates of the organ during treatment. The method comprises directing treatment to at least a portion of an organ in a body part or human or animal subject. The method also comprises imaging multiple two-dimensional images of the organ or surrogates of the organ from varying positions and angles relative to the body part. The method also comprises digitally processing at least a plurality of the multiple two-dimensional images using one or more computers with a software application executing patient-specific, individually trained artificial neural network. The method also comprises displaying estimated three-dimensional motion of the organ or portion of the organ in the body part based on output from the digital processing.

The multiple two-dimensional images may be obtained using a linear accelerator gantry mounted kilovoltage X-ray imager system.

In a fifth aspect, there is provided a method of training a conditional Generative Adversarial Network (cGAN) for determining the position of one or more anatomical objects of interest present in the target area of an image. The method comprises generating a training dataset using Direct Radiograph Rendering (DRRs) from the patients CT images and associated contours at multiple imaging angles at high angular resolution, for example 1 DRR image/0.1°. The method also comprises training a generator network of the cGAN using said training set, where the generator network generates synthetic images of the target area. The method also comprises training a discriminator network of the cGAN to evaluate smaller patches of the synthetic image generated by the generator network, where each evaluation results in a score determining whether the patch is real or fake. The method also comprises calculating an averaged score of all patches evaluated by the discriminator network for each synthetic image. The method also comprises adjusting the generator network based on feedback from the discriminator network to enhance the realism of generated synthetic images. The method also comprises continually optimising both the generator and discriminator networks until no further improvement can be achieved in one network without compromising the performance of the other network.

In a sixth aspect, there is provided an image guidance method for treatment of a predetermined type of organ by a medical device. The method comprises imaging a target area to which the treatment is to be delivered. The method also comprises during the interventional procedure, analysing an image from the imaging with a population-based trained conditional Generative Adversarial Network (cGAN) to determine the position of the predetermined type of organ present in the target area. The method also comprises outputting the determined position(s).

The predetermined type of organ may be any one from the group consisting of: prostate, heart, uterus, kidneys, thyroid and pancreas.

A markerless approach on a conventional radiation therapy treatment system would enable access to real-time IGART for all types of patients without the costs, time, and risks inherent to marker insertion. A trained deep learning model is provided for markerless prostate segmentation in kilo-voltage (kV) X-ray images acquired using a conventional treatment system while the system rotates around the patient, for example, 300 images per revolution. This approach is feasible with kV images acquired for Cone-Beam Computed Tomography (CBCT) (KV-CBCT projection) across an entire radiotherapy arc. Markerless segmentation via deep learning may be useful in various image-guided interventional procedures without the requirements of procuring additional hardware or re-training a highly trained workforce to operate the new functionality provided by the present invention.

Advantageously, there is provided a system for real-time motion monitoring that does not require any additional procedures or hardware. Furthermore, a markerless-based approach using a conventional treatment system would make real-time IGART accessible to all types of patients.

The present invention has industrial application to the analysis of kV images and can be integrated in existing image-guided radiation therapy systems.

According to some embodiments, the cGAN includes a semantic network, for example, a U-Net as the generator, and a CNN as the discriminator.

The present invention enables real-time tracking of the tumour or organ itself during treatment (accommodating for intrafraction motion during radiotherapy, and therefore maintains preciseness for more effective treatments and better outcomes for patients) and is more advantageous than requiring fiducial markers implanted before treatment to be tracked during treatment. In other words, the present invention avoids the risky procedure of having to implant fiducial markers in the patient.

In one embodiment of the present invention, a cGAN is provided which is trained for each patient specifically for their tumour/organ shape using the methodology described to detect the exact shape that had been contoured by a physician prior to treatment, as part of their clinical practice. This is more advantageous than using a convolutional neural network (CNN) approach e.g. semantic segmentation using a U-Net (a type of CNN frequently used in biomedical image segmentation) is risky because the tumour detected by the CNN may not be the same as what the physician had contoured prior to treatment. The cGAN does not suffer from such risk and is therefore more reliable and safer for detecting and tracking the tumour during treatment.

Tumour types can present different levels of difficulty and challenge. For instance, although most prostates are roughly similar in size and shape, most head and neck or lung tumours are not and can vary significantly in size and shape. The full extent of such cancers are not often present radiographically and therefore the approach of using a patient-specific, individually trained conditional Generative Adversarial Network (cGAN) of the present invention, is safer. This is because the cGAN is not a generalised model as it is patient-specific and thus can all shapes and sizes of tumours, especially when these variations are not easily detectable on radiographic images.

The cGAN approach of the present invention enables generation of motion data of the patient at all imaging angles to detect a patient's 6DOF motion. This enables a comprehensive view of a patient's motion during treatment as observed from different imaging perspectives. In contrast, a CNN approach is very limited because they only train a neural network for a specific angle, and therefore is undesirable. If a CNN model does not accurately track the tumour or organ's movement during treatment, it could potentially result in less effective treatment. This is because in radiation therapy, precise targeting of the tumour is crucial to ensure that the radiation dose is maximally delivered to the cancerous cells while minimising exposure to healthy tissues and organs. If the tracking is off, due to limitations in viewing angles, there could be a risk of delivering radiation to healthy tissues (organs at risk) or missing portions of the tumour, reducing the treatment's effectiveness.

The present invention advantageously enables real-time tracking, guidance and position determination of tumours and organs during treatment. This feedback information is provided to the treatment team in the clinic during treatment and does not require any additional hardware or significant retraining of clinic staff. The present invention exceeds the functionality of Kilovoltage Intrafraction Monitoring (KIM) because it has the advantage of not requiring the implantation of fiducial markers before treatment.

The ground truth data needed to train the conditional Generative Adversarial Network is derived from contours created by physicians during routine clinical workflows. These contours are a vital part of the existing treatment planning process, where they delineate the tumour and critical structures within the patient's body to inform and guide therapeutic decisions. This workflow typically involves medical imaging technologies, such as MRI or CT scans, which produce high-resolution images of the patient's internal structures. The physicians then manually define the contours of the tumour and nearby organs on these images. This practice of contouring requires considerable expertise and time as it involves the meticulous tracing of complex anatomical structures. The contours generated in this process are then used as ground truth data to train the cGAN. By feeding this ground truth data to train the cGAN, the model learns to reproduce these contours, thereby learning to identify and track tumours and organs within the patient's body. Using these contours as the ground truth for the cGAN has significant practical benefits. As physicians are already generating these contours as part of standard clinical practice, the present invention leverages and cleverly re-uses this existing resource and eliminates the need for additional annotation work. This is a substantial advantage as manual image annotation is a time-consuming and labour-intensive process, and is often one of the bottlenecks in developing performant machine learning models for medical imaging. By using the contours already created for in existing treatment planning, the present invention does not increase the workload of clinicians and facilitates a more streamlined integration of the cGAN into existing clinical workflows. Furthermore, as these contours are derived directly from the clinical expertise of physicians, they offer a high degree of accuracy and reliability, contributing to the robustness and performance of the cGAN model compared to using a CNN for segmentation.

Unlike CNNs, which require an enormous amount of annotated kV images to train a general model, the cGAN of the present invention leverages the specific patient's data, allowing for a precise, patient-specific model to be generated. This is particularly beneficial because it avoids the need for a vast, generalised training dataset that could potentially introduce noise and irrelevant variations into the model. A CNNs' requirement for a vast amount of annotated ground truth directly on X-ray images is another disadvantage due to the significant time and expense involved. Annotating medical images for machine learning applications is an intensive process that demands a high level of expertise. It often involves medical professionals manually outlining relevant anatomical structures on the images. Given the high cost associated with physicians' time and the vast number of images required, this process for training a CNN can be prohibitively expensive and time-consuming. In contrast, the cGAN approach of the present invention requires significantly fewer annotations as it leverages the contours already created by physicians in the course of clinical treatment planning. This approach not only reduces the cost and time associated with the training process but also enhances the overall efficiency of the model by providing patient-specific, highly relevant training data.

The methodology of the present invention significantly enhances the accuracy and personalisation of cancer treatment by utilising patient-specific data. The present invention uses pre-treatment Computed Tomography (CT) scans and the precise contouring of the tumour by physicians on these scans. The CT scans provide a high-resolution, 3D view of the patient's internal anatomy, thereby giving the cGAN model rich and specific data to work with. The present invention uses Direct Radiograph Rendering (DRR). DRR is a technique that generates X-ray-like images from CT data. DRR is applied at multiple imaging angles to produce a diverse set of images that serve as training data for the cGAN model. These DRR images maintain the fidelity of the original CT scans while providing the necessary variety for robust model training. The generation of these DRR images from multiple angles helps capture the complexity and variability of the human anatomy, and particularly the tumour's characteristics and location. This step trains the network to analyse kV images from multiple imaging angles, which is crucial for the system to track the target in a clinical radiation therapy setting where the treatment is typically a rotational treatment such as IMRT or VMAT treatments. Thus, multiple-angle DRR information is vital in ensuring accurate tracking, monitoring, and treatment during the radiation therapy sessions. Training a patient-specific cGAN model using this method represents a substantial improvement over traditional Convolutional Neural Networks (CNNs). As opposed to generalised models that CNNs produce, a cGAN model trained on patient-specific data, particularly with multiple-angle DRRs, is more capable of accurately capturing the patient's unique anatomy and the specifics of the tumour.

The methodology of the present invention is not limited to adversarial learning or specifically, cGANs. It can be applied to any type of AI training that requires patient-specific information which would result in better performance compared to CNNs. Adversarial learning has useful characteristics for training an artificial neural network of the present invention, as it involves a competitive dynamic (i.e. between a generator and a discriminator), is unsupervised (learning through mimicking), is generative (can create synthetic data resembling input data) and has implicit loss functions defined by the discriminator's ability to distinguish between real and fake data. Other types of AI training envisaged include transfer learning, few-shot learning, multi-task learning or AutoML systems.

Advantageously, the present invention includes motion (as an element of real-world conditions) in the training data for the cGAN to augment the training data. A patient's body, organs, or the tumour itself may move due to breathing, peristalsis, or other natural physiological processes. By accounting for these movements, the present invention delivers a more accurate and realistic representation of the treatment environment. The inclusion of motion in the training data essentially augments the data set, expanding the range of situations the conditional Generative Adversarial Network (cGAN) model might encounter during treatment. This augmentation is achieved by simulating various types of motion in the patient's body and incorporating these variations into the training data. Such simulations might include movements due to breathing cycles or shifts in organ positions. This expansion of the training data is vital for creating a robust and accurate cGAN model. Not only does it improve the model's ability to track and monitor the tumour during treatment, but it also enhances its capability to account for and adapt to changes in the patient's body. As a result, the trained model can accurately predict the location of the tumour despite body movements, leading to more effective treatment delivery and minimised risk of damaging healthy tissues. This approach significantly surpasses some traditional training methods, which may have overlooked the dynamic nature of a living body. By incorporating motion into the training data, the present invention ensures that the resulting cGAN model is better equipped to handle the complexities of real-world conditions during cancer treatment.

Rather than using generalised data from a plurality of patients, the present invention uses the patient CT and the tumour/organ contour in 3D by the physician on the pre-treatment CT to train the cGAN model for the patient. This ensures a highly personalised and accurate model and involves using a pre-treatment CT (Computed Tomography) scan of the patient and physician-drawn contours of the tumour or organ in question. The CT scan provides high-resolution three-dimensional images of the patient's body, offering valuable details about the shape, size, and location of the tumour or the organ. It serves as comprehensive and detailed training data for the cGAN model, enabling it to accurately understand the patient's unique anatomy and the specific characteristics of the tumour or organ. Whereas the tumour/organ contour drawn by the physician offers essential information about the precise boundaries and shape of the tumour or organ. These contours, drawn on the 3D pre-treatment CT images, provide the exact shape that the physician has identified as the treatment target. This personalised contouring provides an accurate representation of the target area, facilitating precise treatment planning and execution. Training the cGAN model using these personalised data ensures that the model is highly accurate and specific to each patient. It essentially tailors the model to each individual's unique physiological characteristics, enhancing the accuracy of tumour tracking and treatment delivery. This is advantageous over approaches that rely on generalised models, which might not account for individual patient variations and could lead to less accurate treatment delivery. The cGAN model of the present invention aligns well with the patient's unique body structure and the specific characteristics of the tumour or organ, thereby improving the accuracy of real-time tracking during treatment. This results in more effective cancer treatment, with less risk of damaging healthy tissues around the target area.

As used herein, except where the context requires otherwise, the term “comprise” and variations of the term, such as “comprising”, “comprises” and “comprised”, are not intended to exclude further additives, components, integers or steps.

Further aspects, advantages, and features of embodiments of the invention will be apparent to persons skilled in the relevant arts from the following description of various embodiments. It will be appreciated, however, that the invention is not limited to the embodiments described, which are provided in order to illustrate the principles of the invention as defined in the foregoing statements and in the appended claims, and to assist skilled persons in putting these principles into practical effect.

### BRIEF DESCRIPTION OF THE DRAWINGS

Embodiments of the invention will now be described with reference to the accompanying drawings, in which like reference numerals indicate like features, and wherein:

FIG. 1 is a flow chart of a clinical workflow using a conditional Generative Adversarial Network (cGAN) model in accordance with an embodiment of the present invention for cancer target tracking during radiation therapy treatment.

FIG. 2 is a flow chart of data generation, training and evaluation phases for a conditional Generative Adversarial Network (cGAN) model in accordance with an embodiment of the present invention for prostate cancer.

FIG. 3 is a plurality of boxplots comprising centroid tracking error results (FIG. 3a and FIG. 3b), and DSC results: DICE coefficient of the tracked target vs ground-truth (FIG. 3c and FIG. 3d).

FIG. 4 is a series of X-rays depicting an example of the method in accordance with an embodiment of the present invention applied to prostate cancer and shows the cGAN segmentations.

FIG. 5 is a flow chart showing the method in accordance with an embodiment of the present invention used to evaluate the patient-specific deep learning method for a single patient that has head and neck cancer.

FIG. 6 is a series of violin plots showing the distribution of the accuracy metrics for cGAN segmentation (blue) compared with the no-tracking segmentations (orange) for the different tumour locations. The metrics shown are the magnitude of the absolute centroid error (top), the Dice Similarity Coefficient (middle) and mean surface distance (bottom). The width of the violin plot at each y value corresponds to the frequency of that value. Tracking accuracy of the method for head and neck cancer on the evaluated cohort can be observed from the violin plots.

FIG. 7 is a series of X-ray images depicting cGAN segmentation (red) and the no-tracking segmentation (blue) in comparison to the ground truth segmentation (green) for different projection angles, which are tracking a head and neck cancer tumour.

FIG. 8 depicts a series of violin plots of Simultaneous Tumour and Organs at Risk Tracking (STOART) segmentation results in comparison to the ground truth segmentation of the tumour and the heart in kV-projections from seven lung cancer patients, showing the method in accordance with an embodiment of the present invention for simultaneous tracking of the heart and lung tumour. The violin plots show the Dice-similarity-coefficient (DSC) and mean surface distance for the tumour and heart segmentation compared to the ground truth for all kV projections. The white dot and line indicate the mean and standard deviation of the error, respectively. The width of the violin relates to the number of data samples for a given value.

FIG. 9 is an architecture diagram of the cGAN depicting the architecture of the generator network (top) and the discriminator network (bottom).

FIG. 10 is a block diagram illustrating a schematic representation of a system configured to implement an embodiment of the present invention.

FIG. 11 is a workflow diagram depicting the three phases of dataset creation, model training and application of the trained model in accordance with an embodiment of the present invention for segmenting the tumour and the heart in cone beam computed tomography (CBCT) projection.

### DETAILED DESCRIPTION OF EMBODIMENTS

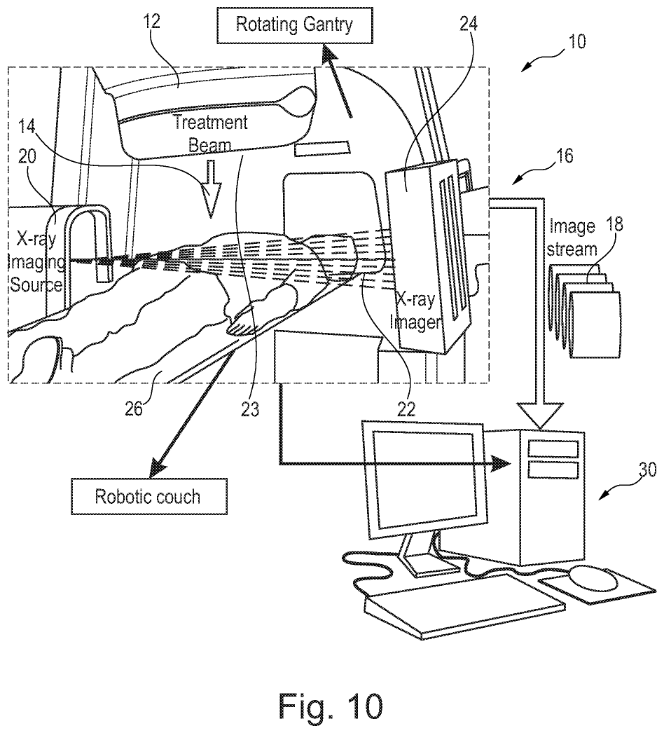

FIG. 10 depicts a system for image guided radiation therapy that may implement an embodiment of the inventions described herein.

The system 10 includes a radiation source 12 for emitting at least one treatment beam of radiation. The radiation source emits the treatment beam 14 along a first beam axis towards the patient being treated. Typically the radiation source 12 will comprise a linear accelerator emitting megavolt X-rays.

The system 10 also includes an imaging system 16 arranged to generate a succession of images 18 comprising a two-dimensional projection of a field of view and in which the location of the target may be identified. The imaging system 16 includes a second radiation source 20 that emits at least one imaging beam 22 along a second beam axis. The imaging beam 22 will be transmitted in a direction orthogonal to the treatment beam 14. The imaging beam is transmitted through the patient (or at least through the region of the patient) to a radiation detector 24 that is configured to detect radiation transmitted through the target. The spatial intensity of the received radiation is converted to an X-ray image that is a projection of said at least one imaging beam in a plane normal to the direction its emission. Typically, the imaging system 16 will be a kilovoltage (kV) imaging system 16 built into the linear accelerator 12. In embodiments of the present invention, the imaging system 16 is arranged to only intermittently emit its imaging beam to thereby reduce the patient's radiation exposure compared to continuous imaging. The rate of imaging can vary depending on requirements or system configuration but will typically have an imaging interval between 0.1 s to 60 s. Some embodiments may have a longer or shorter imaging interval.

The system 10 also includes a support platform 26 (e.g. a bed) on which the subject of the radiation therapy is supported during treatment. Support platform 26 is repositionable relative to the imaging system 16 and radiation source, so that the patient can be positioned with the centre of the target (e.g. tumour) located as near as possible to the intersection between the first and second beam axes.

The system 10 may also include a respiratory monitor 23 which generates a signal indicative of the respiration of the patient. In some instances, system 10 does not require a respiratory monitor 23 such as for prostate treatment. The respiratory monitor 23 can generate a continuous signal from external surface or volumetric signal. The source of the external signal can come from either optical surface monitoring devices such as RPM (Varian), AlignRT (VisionRT), or volumetric measurements such as the bellows belt (Philips Healthcare).

The system 10 also includes a control system 30 that controls the parameters of operation of the radiation therapy system. Generally speaking, the control system 30 is a computer system comprising one or more processors with associated working memory, data storage and other necessary hardware, that operates under control of software instructions to receive input data from one or more of a user, other components of the system (e.g. the imaging system 16), and outputs control signals to control the operation of the radiation therapy system. Amongst other things, the control system 30 causes the radiation source 12 to direct its at least one treatment beam at the target. To do this, the control system 30 receives images from the imaging system 16, analyses those images to determine the position of fiducial markers present in the target (thereby estimating the motion of the target), and then issues a control signal to adjust the system 10 to better direct the treatment beam 14 at the target.

As will be appreciated by those skilled in the art, the radiation source 12, imaging system 16 and support platform 30 are common to most conventional image radiation therapy systems. Accordingly, in the conventional manner the radiation source 12 and imaging system 16 can be rotatably mounted (on a structure commonly called a gantry) with respect to the patient support platform 30 so that they can rotate about the patient in use. The rotational axis of the gantry motion is typically orthogonal to the directions of the treatment beam and imaging beam (i.e. the first and second directions.) This enables sequential treatment and imaging of the patient at different angular positions about the system's gantry's axis.

As noted above, the control system 30 processes images received from the imaging system 16 to estimate the motion of the target, and then issues a control signal to adjust the system 10 to direct the treatment beam at the target better. The adjustment typically comprises at least one of the following: changing a geometrical property of the treatment beam such as its shape or position, e.g. by adapting a multi-leaf collimator of the linear accelerator (linac); changing the time of emission of the beam, e.g. by delaying treatment beam activation to a more suitable time; gating the operation of the beam, e.g. turning off the beam if the estimated motion is greater than certain parameters; changing an angle at which the beam is emitted relative to the target about the system rotational axes. The system 10 can also be adjusted to better direct the treatment beam at the target by moving the patient support platform 26. Moving the support platform 26 effectively changes the position of the centroid of the target with respect to the position of the treatment beam 14 (and imaging beam).

In use, the general method of operation of the system 10 is as follows. The radiation source and imaging system 16 rotates around the patient during treatment. The imaging system 16 acquires 2D projections of the target separated by an appropriate time interval. The control system 30 uses the periodically received 2D projections (e.g. kV X-ray images) to estimate the target's position. The control system 30 therefore needs a mechanism for determining the position of the target and then performing ongoing estimation of the target's location and orientation in 3-dimensions.

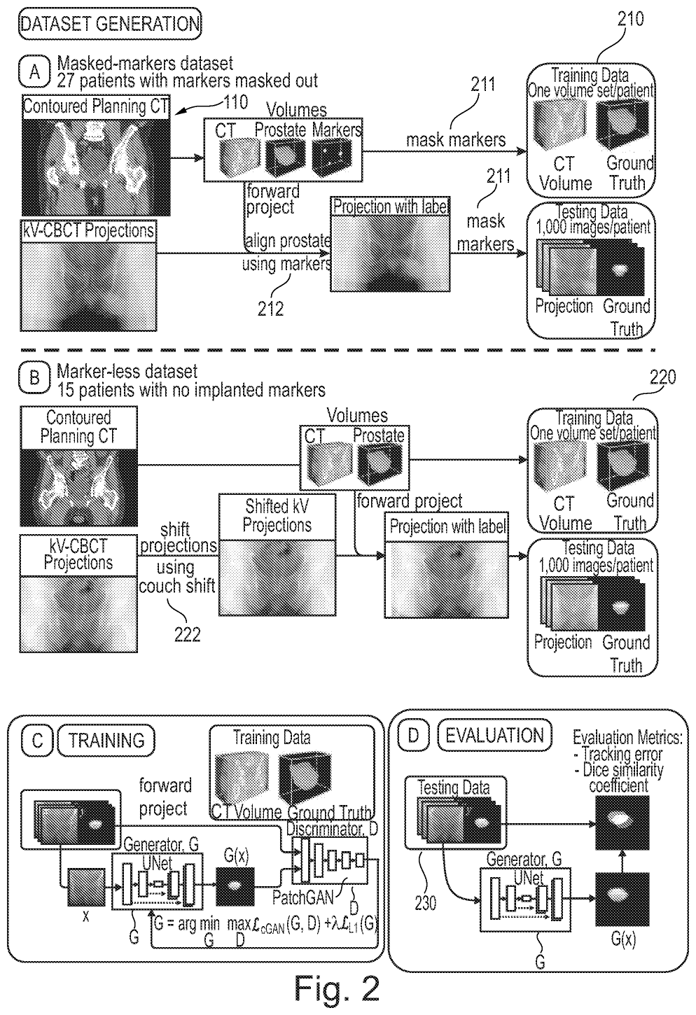

FIG. 2 illustrates a method of guided radiation therapy in which the present invention can be practiced. The methods of guided radiation therapy are similar to those followed by Huang et al. 2015 (Huang, C.-Y., Tehrani, J. N., Ng, J. A., Booth, J. T. & Keall, P. J. 2015. SixDegrees-of-Freedom Prostate and Lung Tumour Motion Measurements Using Kilovoltage Intrafraction Monitoring. Int J Radiat Oncol Biol Phys, 91, 368-375;); and Keall et al. 2016 (Keall, P. J., Ng, J. A., Juneja, P., O'brien, R. T., Huang, C.-Y., Colvill, E., Caillet, V., Simpson, E., Poulsen, P. R., Kneebone, A., Eade, T. & Booth, J. T. 2016. Real-Time 3D Image Guidance Using a Standard LINAC: Measured Motion, Accuracy, and Precision of the First Prospective Clinical Trial of Kilovoltage Intrafraction Monitoring Guided Gating for Prostate Cancer Radiation Therapy. Int J Radiat Oncol Biol Phys, 94, 1015-1021) (the contents of which are each incorporated by reference for all purposes with the exception of the use of the markerless real-time motion tracking method described herein).

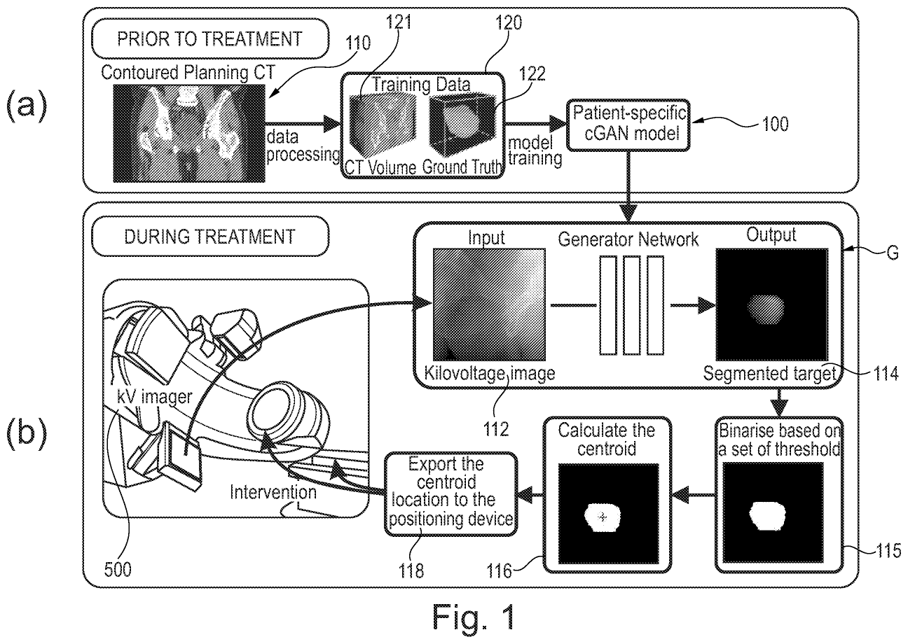

At least one embodiment of the present invention provides markerless prostate segmentation performed in kV cone-beam computed tomography (KV-CBCT) projections 112 with a patient-specific prior by leveraging deep learning. Referring to FIG. 1, clinical implementation of prostate motion monitoring through tracking the prostate in kV-CBCT projections using deep learning-based image to image translation is provided. The method uses a patient-specific model 100 (the model is specific to an individual patient) that is trained on the three-dimensional (3D) planning CT and prostate contour 110 that can be incorporated into a treatment workflow, as shown in FIG. 1. A conditional Generative Adversarial Network (cGAN) 100 is used to segment the prostate in two-dimensional (2D) kV-CBCT projections 112. It is advantageous to use a patient-specific model as it requires less data than training a generalised model. Furthermore, it enables the model 100 to learn features most relevant to the specific patient under treatment and can be applicable to a diverse range of patients imaged using different imaging systems. The approach leverages the patient's own imaging and planning data that is available prior to the commencement of their treatment. The model 100 can be evaluated using imaging data both with and without markers from four different clinical sites in Australia. The results indicate that the prostate can be tracked with a high degree of accuracy.

Referring to FIG. 1, the clinical workflow for automatic prostate tracking comprises two key components: prior treatment (FIG. 1a) and during treatment (FIG. 1b). A patient-specific network 100 is trained prior to the patient's treatment using digitally reconstructed radiograph (DRR) data. The generator network G from the cGAN 100 is used during the treatment to segment the target. The location of the segmented target 114 can be used for motion management.

Referring to FIG. 2, two separate datasets 210, 220 are used to evaluate the performance of the cGAN segmentation: a masked-markers dataset (FIG. 2a) and a markerless dataset (FIG. 2b). The ground truth is generated through a unique approach for each dataset 210, 220 since annotation by clinicians is not feasible due to the low contrast of the kV-CBCT projections.

In FIG. 2a, the masked-markers dataset 210 is generated from imaging data of prostate cancer patients with implanted fiducial markers. The markers are used to align the ground truth contour and are masked out in order to not bias the model during training. To obtain the ground-truth position of the prostate, the markers are used to annotate the real-time location of the prostate in the kV-CBCT projections. During testing of the model 100, the real-time prediction of the prostate location could be compared with the ground-truth prostate location, using the implanted markers as surrogates. The fiducial markers are masked out in the training and testing data to avoid biasing the deep learning model. The masking algorithm is based on morphological reconstruction, with Poisson noise applied. Visual inspection of each kV-CBCT projection is performed to ensure that the markers are sufficiently masked and no longer visible.

In FIG. 2b, the markerless dataset 220 is generated from imaging data of prostate cancer patients with no implanted fiducial markers. The kV-CBCT projections are shifted based on the couch shift for each fraction. The kV-CBCT projections are shifted based on image registration performed between the treatment CBCT and planning CT. Therefore, the ground truth in the markerless dataset 220 is defined by the average location of the prostate rather than the real-time location.

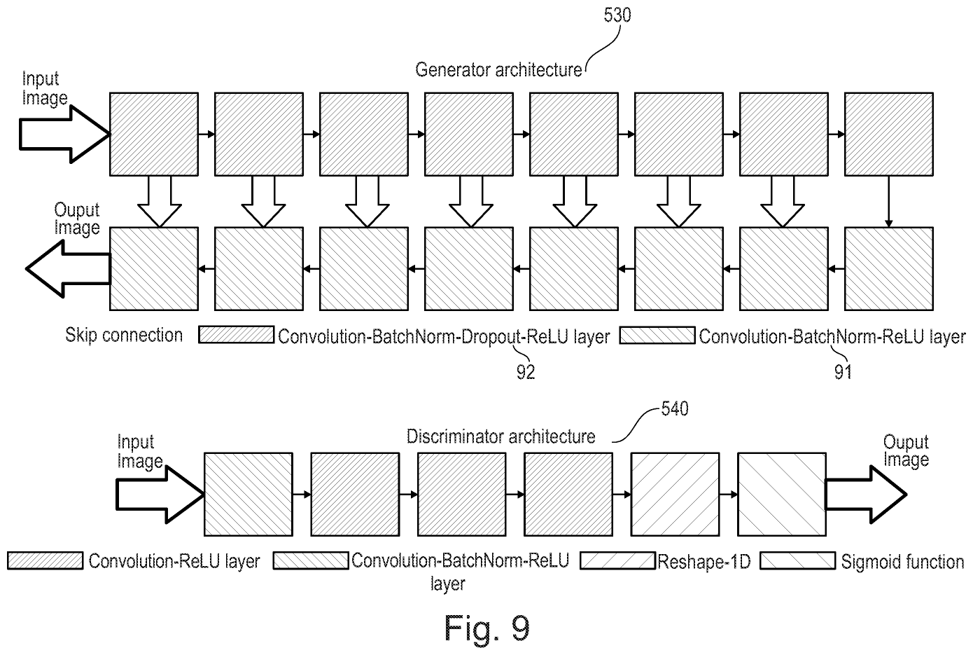

In FIG. 2c, the data is used to train a cGAN model 100 for each patient consisting of a U-Net generator network, G, and a PatchGAN discriminator network, D. For this approach to be clinically feasible, the patient-specific model 100 must be trained using data available prior to the patient's first treatment. In the clinical workflow, a patient will have a planning CT several weeks prior to the first treatment which is used by clinicians to contour the relevant volumes. Therefore, this available data is used to train the model prior to the patient's first treatment. The inputs to the model are the planning CT and prostate contour 3D volumes. The volumes are forward projected to digitally produce 2D projections every 0.1 degree over 360 degrees, generating 3,600 projections. The projections produced from the planning CT are each paired with the respective projection produced from the prostate contour. A cGAN 100 is applied to segment the prostate from 2D kV-CBCT projections. Details about the network architecture are depicted in FIG. 9.

In FIG. 2d, the cGAN 100 model is evaluated using the testing data 230 and the performance is quantified using the centroid tracking error and DSC. KV-CBCT projections are evaluated from two fractions of each patient's treatment.

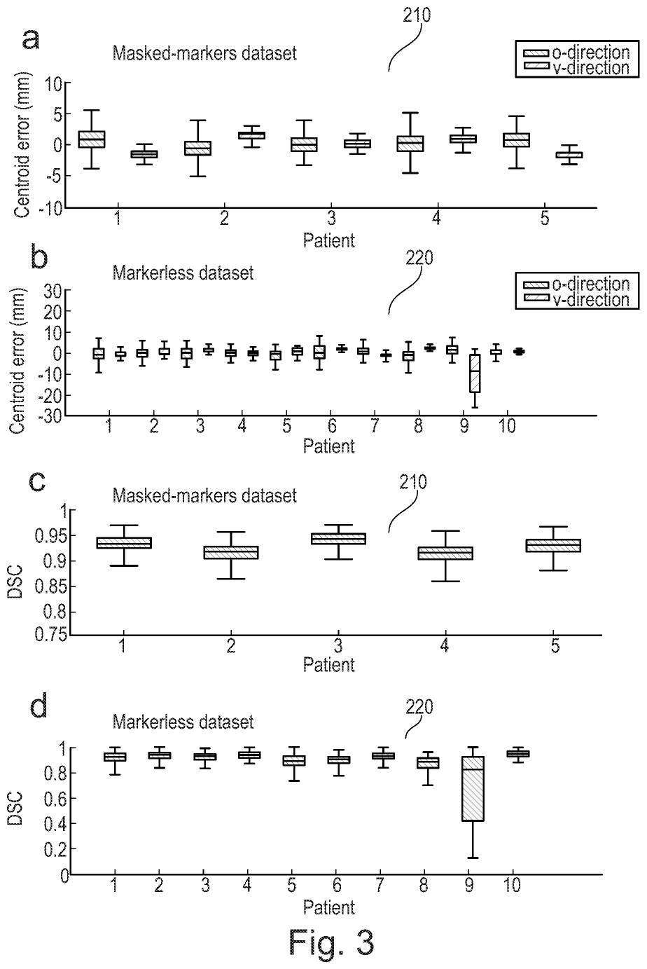

The centroid errors of the cGAN segmentations for all patients are represented in FIG. 3. The centroid errors are presented in the kV-CBCT frame of reference where the u-direction represents the lateral and anterior-posterior directions and the v-direction represents the superior-inferior directions. For the masked-markers dataset 210, there is a slight offset in both the u- and v-directions of 0.2 mm and −0.2 mm, respectively. The tracking system 10 had higher accuracy in the v-direction (FIG. 3a). The 2.5th and 97.5th percentiles are both under 3 mm in the v-direction, while the percentiles are over 3 mm in the u-direction. Similar performance is observed for the markerless dataset 220 with the exception of patient 9. The overall error is −0.1±2.7 mm and 0.1±1.5 mm in the u- and v-directions, respectively.

Boxplots of the centroid tracking error results in FIG. 3a and FIG. 3b. The errors in the centroid location of the cGAN segmentation versus the ground truth for each patient in the u- and v-directions for the masked-markers dataset (FIG. 3a) and markerless dataset (FIG. 3b)

The present invention provides real-time, markerless prostate tracking during treatment with high accuracy, leveraging a patient-specific deep learning model 100. In a preferred embodiment, the model is a cGAN. This model 100 was evaluated using data sourced from a conventional radiation therapy treatment system spanning multiple treatment sites. The performance of the cGAN 100 was evaluated by assessing the predicted target volume's union with the ground truth, otherwise known as the Dice Similarity Coefficient (DSC). This was done for both the masked-markers dataset (FIG. 3c) and the markerless dataset (FIG. 3d). In both datasets 210, 220, a high agreement between the predicted and ground truth segmentations was observed, with an overall mean DSC of 0.92±0.03 and 0.91±0.05 for the masked-markers and markerless dataset, respectively. Patient 9 in the markerless dataset 220 emerged as an outlier, as observed in the centroid error (FIG. 3d).

The cGAN's performance is very fast, with the trained network generating the segmentation (i.e., the inferencing time) in approximately 0.01 seconds per image. This speed is advantageous during treatment as it allows real-time monitoring with extended duration, such as hypofractionated SBRT.

The tracking accuracy of the model 100 in the masked-markers dataset was 0.2±1.9 mm and −0.2±1.5 mm in the u- and v-directions respectively, with similar accuracy observed in the markerless dataset (0.1±2.7 mm and 0.1±1.5 mm in the u- and v-directions, respectively). Studies by Langen et al. and Su et al. highlighted that prostate displacement can increase over time. Therefore, the need for real-time motion monitoring becomes pivotal which the present invention has largely addressed.

The model 100 is insensitive to motion in the plane perpendicular to the detector plane as it estimates position in the 2D KV-CBCT projection frame of reference. For clinical applications, an algorithm will need to be implemented to infer the 3D target coordinates from these 2D projections, a technique already used for marker-based tumour tracking. Various successful estimation methods such as a 3D Gaussian PDF, Bayesian inference, or a Kalman filter may be adapted using the segmentation boundary or centroid for this approach. While the reported accuracy is in 2D, it is reasonable to expect that the model 100 is capable of detecting high motion cases, given the high mean DSC on both datasets. This indicates potential for gating when a defined percentage of the prostate moves outside the defined treatment boundary.

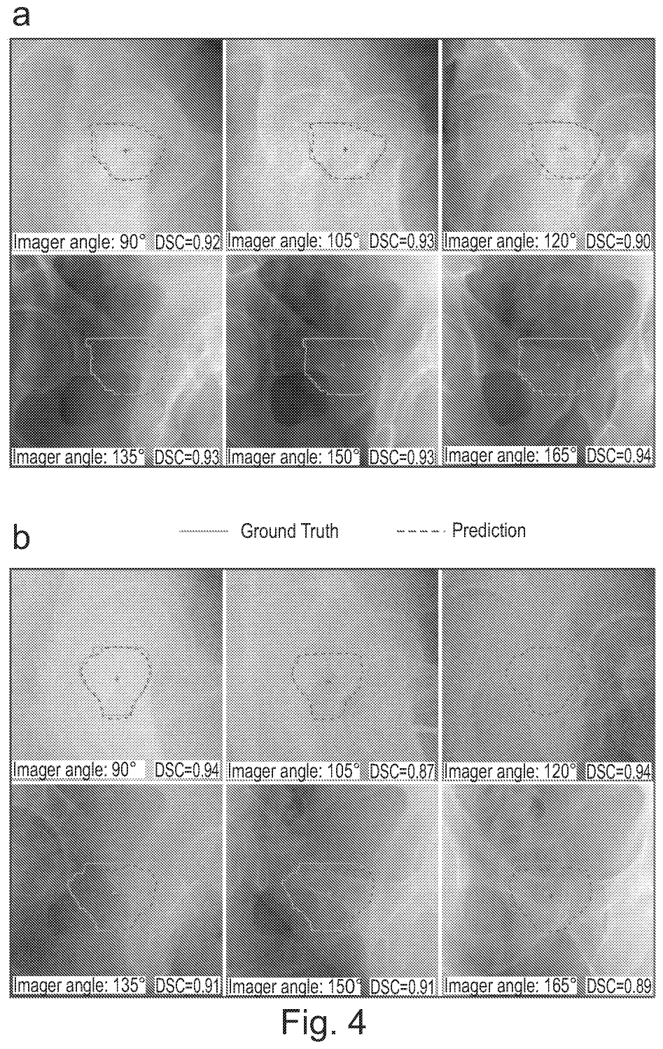

An example of the cGAN segmentations at different projection angles (e.g. six angles) are shown in FIG. 4 which demonstrates an example of the typical accuracy of the cGAN segmentations achieved by the present invention. FIG. 4a depicts segmentations from fraction 1 of patient 3 in the masked-markers dataset. FIG. 4b depicts segmentations from fraction 1 of patient 4 in the markerless dataset.

Current methods for tracking the prostate during radiation therapy rely on implanted fiducial markers. The accuracy of marker-based methods is the gold standard for markerless methods because marker-based methods can achieve sub-millimetre accuracy. However, markerless tracking of the prostate and pancreas is more difficult than other sites due to the lower soft tissue contrast. MRI-linacs are an alternative for IGART for the prostate and pancreas due to improved soft tissue contrast compared to kV projections. However, MRI-linacs are an expensive treatment option for patients and are not widely available compared to the greater availability of standard linacs. Deep learning approaches for markerless MRI-guided radiotherapy have typically focused only on lung and liver tracking.

The model 100 of the present invention has several features that make it ideal for clinical implementation. First, the model 100 takes 3 hours on average to train (using a workstation with a NIVIDA Quadro P6000 GPU) which makes it feasible for patient-specific training (i.e. training a model for an individual patient) in between the patient's planning session and their first treatment. Second, the inference time of the model is 0.01 second on average per image. This makes the model suitable for real-time applications as the American Association of Physicists in Medicine (AAPM) Task Group 264 defines real-time as a system latency below 0.5 second. A single network approach for tracking at all angles across the entire treatment arc is less computationally intensive than using several networks for distinct angles. Third, the model produces a segmentation of the prostate which can be beneficial for other applications such as real-time dose accumulation. Finally, the model 100 is patient-specific, enabling it to learn features relevant to the individual patient and imaging system 16 used during treatment. The robustness of health AI algorithms is a major concern for regulators and the medical community, and the present invention ameliorates this concern effectively. Often the performance of an algorithm can be correlated to the particular data used for training. However, this is not a concern in the present invention because each patient specific model 100 has been successfully tested across four different clinical sites, achieving a similar performance across all patients treated.

There may be uncertainties related to the ground truth of the prostate in the kV-CBCT projections. Due to the low soft tissue contrast in the kV-CBCT projections, it is not possible to manually contour or label the prostate. Therefore, other solutions are required to generate the ground truth. Two methods of producing the ground truth are described. Results from both methods together are intended to reduce the uncertainties related to the results. The masked-markers dataset 210 is generated from imaging data of prostate cancer patients with implanted fiducial markers. The markers are used to annotate the real-time location of the prostate in the kV-CBCT projections. However, fiducial markers are subject to surrogacy errors and may therefore limit the accuracy of the ground truth prostate segmentation. The markerless dataset 220 is generated from prostate cancer patients' imaging data with no implanted fiducial markers. Since it is not possible to annotate the real-time location, the kV-CBCT projections are shifted 222 based on image registration performed between the treatment CBCT and planning CT giving an average location. The lack of a real-time ground truth may be partly responsible for the increase in tracking uncertainty compared to the masked-marker dataset 210. The results from these two approaches provide increased certainty of the model performance for markerless tracking.

Another limitation may be that the model is evaluated using kV-CBCT projections rather than intrafraction kV projections. The quality of the ground truth is prioritised and hence used kV-CBCT projections for this embodiment. To generate the ground truth for the markerless dataset 220, image registration is required between the treatment 3D CBCT and planning CT to determine the average location. While CBCT projections provide superior quality to kV projections, state-of-the-art clinical systems provide solutions to minimise the effect of MV scatter and provide an improved kV projection quality. One such solution is trigged imaging that is incorporated into Varian systems. Triggered imaging improves kV projection quality by placing the treatment beam on hold prior to acquisition of each triggered image in order to eliminate the effect of MV scatter. Frame averaging has been previously used to reduce noise in the projections. As the model 100 is trained on a case-by-case basis, it can benefit from any improvement in kV projection quality that may occur over time.

Clinical implementation of real-time markerless prostate tracking throughout radiation therapy is provided and demonstrates the potential to be expanded to other soft-tissue organs such as the pancreas, liver, and kidneys. The approach only requires projections from the On-Board Imaging system (OBI) which includes linear accelerators from Elekta, Varian, and other manufacturers. The markerless method can lead to greater access to IGART treatments for all types of patients, eliminating the time delays, costs and risks associated with the requirement of fiducial markers being implanted into the patient prior to treatment.

Masked-markers dataset. The masked-markers dataset 210 is generated using imaging data of patients with implanted fiducial markers, which are masked-out 211 for training and analysis (FIG. 2a). The dataset 210 is constructed using the imaging data of 27 prostate cancer patients undergoing radiation therapy in the TROG 15.01 SPARK trial. The patients for this study were treated on the Varian TrueBeam at different sites. The planning CT, physician contours, and kV-CBCT projections were collected from two fractions associated with this cohort. 500 KV-CBCT projections are used from each fraction, giving a total of 27,000 kV-CBCT projections. Each patient has three cylindrical gold fiducial markers implanted in their prostate. The training data 120 is the 3D planning CT and prostate contour. The test data is generated using the kV-CBCT projections from two fractions. The prostate contour volume 121 is projected as a 2D digitally reconstructed radiographs (DRR) for each kV-CBCT projection angle. The DRRs are generated using the Reconstruction Toolkit and the Insight Toolkit. The ground truth 122 is generated by aligning 212 the prostate-only DRR with the kV-CBCT projections based on the implanted fiducial markers. Following alignment, the fiducial markers are masked out 211 in all DRRs and CT projections to avoid biasing the model. The masking algorithm is based on morphological reconstruction with Poisson noise applied 520. Markerless dataset. The markerless dataset 220 is generated using imaging data of patients with no implanted fiducial markers (FIG. 2b). The dataset 220 is constructed using the imaging data of ten prostate cancer patients undergoing radiation therapy in the OPTIMAL trial (NCT03386045). Patients were treated on the TrueBeam STx (Varian Medical Systems, Palo Alto) Linac. The planning CT, physician contours, and kV-CBCT projections are collected from two fractions associated with this cohort. 500 KV-CBCT projections are used from each fraction, providing a total of 15,000 kV-CBCT projections. The training data 220 is generated in the same way as the masked-markers dataset 210. Since these patients did not have implanted fiducial markers, the ground truth is generated using shifts based on image registration performed between the treatment CBCT and planning CT. The 2D kV-CBCT projections are shifted 222 based on the 3D couch shift using equation 1.

$$\hat{P} ( \hat{M} ( t ) \vert θ ) = ( \begin{matrix}\hat{u} \\ \hat{v}\end{matrix} ) = \frac{SID}{SAD - ( \hat{x} \cdot cos θ + \hat{z} \cdot sin θ )} ( \begin{matrix}\hat{x} \cdot sin θ - \hat{z} \cdot cos θ \\ \hat{y}\end{matrix} ) \qquad ( 1 )$$

where SID is the linac source-isocentre distance, SAD is the linac source-aperture distance.

After shifting the kV-CBCT projections, prostate-only DRRs are generated for each kV-CBCT projection angle. Therefore, the ground truth in the markerless dataset 220 is defined by the average location of the prostate instead of the real-time location.

cGAN framework. The tracking system 10 uses a cGAN model 100 for segmentation of the prostate. The training of the model 100 involves adversarial learning between the generator network G and the discriminator network D. The cGAN model 100 is trained to replicate a prostate segmentation given a pelvis projection as input. The generator G takes the input projection 112 and creates a segmentation image 114. The discriminator D classifies whether the paired image discriminator network D comes from the training set or the generator network G as shown in FIG. 2c. The cGAN 100 is initialised with random parameters and trained to minimise the loss function:

$${G}^{*} = arg {min}_{G} {max}_{D} {ℒ}_{cGAN} ( G , D ) + λ {ℒ}_{L 1} ( G ) \qquad ( 2 )$$

where λ is a constant (set to 100 for this implementation) and:

$${ℒ}_{cGAN} ( G , D ) = {E}_{x , y} [ log D ( x , y ) ] + {E}_{x} [ log ( 1 - D ( x , G ( x ) ) ) ] \qquad ( 3 )$$

$${ℒ}_{L 1} = {E}_{x , y} {‖ y - G ( x ) ‖}_{1} \qquad ( 4 )$$

In one embodiment, the cGAN implementation is based on the Pix2pix model. A 70×70 PatchGAN is used for the discriminator architecture D and 256×256 U-Net 530 for the generator architecture G. The inputs are two volumetric images: the planning CT and prostate contour volumes. The volumes are forward projected to digitally produce 2D projections every 0.1 degree over 360 degrees, generating 3,600 projections using the Reconstruction Toolkit (RTK) and Insight Toolkit (ITK). The projections produced from the planning CT are each paired 522 with the respective projection produced from the prostate contour. The projections of size 550×550 pixels are cropped 525 to 512×512 pixels. The crop position is randomly shifted, resulting in a maximum motion of 10 mm to replicate or simulate possible treatment setup error and anatomical motion. Each model 100 is trained for ten epochs with a batch size of four and a learning rate of 0.0002 using the Adam optimiser, a stochastic gradient descent method. The models 100 are trained on a computer with an Intel® Xeon® Gold 6248R processor (3.0 GHZ), as the Central Processing Unit (CPU), with 256 GB RAM, as memory, and a NIVIDIA® RTX A6000 Graphics Processing Unit (GPU).

Referring to FIG. 9, the architectural structures of both the generator (U-Net) 530 and discriminator (PatchGAN) 540 networks implemented in the cGAN model 100 are outlined. The generator network G and the discriminator network D are important components of the cGAN model, functioning collaboratively to achieve high fidelity output.

The generator network G is structured with layers that incorporate a series of operations: Convolution-BatchNorm-Dropout-ReLU 92. In these layers 92, the Rectified Linear Unit (ReLU) activation functions are leaky. This means that instead of the function output being set to zero when the input is negative, a small, non-zero gradient (in this example, a slope of 0.2) is allowed. This feature helps mitigate the issue of dying ReLUs, where neurons effectively become inactive and only output zero, limiting the network's capacity to learn. The generator network G also incorporates Convolution-BatchNorm-ReLU layers 91, where the ReLUs are not leaky. For negative input values, these ReLU functions will output zero, following a traditional ReLU activation function approach.

The discriminator network D is this example is a PatchGAN 540, and uses a novel method to assess whether an image is real or fake. Instead of making a single determination for the entirety of the image, it applies its discriminative judgement across 70×70 patches convolutionally. The discriminator D scans over the image, evaluating smaller patches separately to decide whether each 70×70 patch is real or fake. This enhances the efficiency of the discriminator D and also allows it to focus on local image characteristics. After evaluating all the patches, the PatchGAN 540 derives an averaged output over all the 70×70 patches. This final score reflects the discriminator's assessment of the image's authenticity. As a result, this method provides a more nuanced and detailed determination of the generated images, boosting the overall performance and effectiveness of the cGAN model 100.

Although training of the network should stop when the loss function reaches a minimum, given the adversarial training approach used when training a GAN, convergence is achieved when an equilibrium between the two adversarial networks G, D is reached, where one network can't be improved without compromising the other competing network. After two epochs the cGAN loss increases, but at the cost of the Discriminator network, shown by the inconsistent performance of the D_fake loss metric. This implies that one network has improved by worsening the other network, which means the networks aren't in equilibrium. In contrast, epoch 12 shows the loss functions in equilibrium showing that D_real≈D_Fake, as well as a stable cGAN loss, similar to stopping criteria that has been used previously.

Clinical application. In one embodiment, the cGAN model 100 can be incorporated into the treatment workflow for intrafraction monitoring of the prostate in fluoroscopic images 112 (see FIG. 1b). For clinical implementation, the central component of the workflow is the deep learning model G for prostate segmentation. The generator network G in the cGAN produces a prediction image 114 based on a fluoroscopic input image 112 for the prostate segmentation. The prediction image 114 is binarised 115 using a set threshold value to give the segmentation, and the centroid of the segmentation is calculated 116. If multiple unconnected regions are detected, the centroid of the most significant region is calculated 116. The calculated centroid location can be exported 118 to the positioning systems to enable real-time motion adjustments during the treatment of the patient.

Analysis. The models are tested on the unseen kV-CBCT projections to evaluate the accuracy of the prostate segmentation and the tracking system. The models are tested using the kV-CBCT projections from two fractions of each patient, giving 1,000 test images per patient (500 per fraction). The cGAN segmentation is binarised based on a 0.1 threshold. The cGAN segmentation is compared to the ground truth segmentation for the analysis. The generator's ability to produce accurate prostate segmentations is evaluated for each patient model. The performance is quantified by calculating the DSC, which gauges the similarity of the two prostate segmentations based on the overlap. If multiple unconnected regions are present in the cGAN segmentation, the DSC is calculated using the largest region. Finally, the generator's ability to be used in an automated tracking system is evaluated by using the centroid of the segmentations. If multiple unconnected regions are present in the cGAN segmentation, the centroid is calculated using the largest region. The tracking system error is defined as the cGAN segmentation centroid minus the ground truth segmentation centroid. The errors are calculated in the lateral/anterior-posterior (LR/AP) and superior-inferior (SI) directions. The errors are reported at the patient coordinate system using the source-isocentre distance/source-detector distance ratio (=1.5) as correction factor. The overall errors are quantified by calculating the mean error and the 2.5^{th} and 97.5^{th} percentiles of the errors.

### Example—Head and Neck Tumours

Using radiation therapy (RT) to treat head and neck (H&N) cancers requires precise targeting of the tumour to avoid damaging the surrounding healthy organs. Immobilisation masks and planning target volume margins are used to attempt to mitigate patient motion during treatment, however patient motion can still occur. Patient motion during RT can lead to decreased treatment effectiveness and a higher chance of treatment related side effects. Tracking tumour motion would enable motion compensation during RT, leading to more accurate dose delivery.

Radiation therapy (RT) is indicated for 74% of H&N cancer patients. H&N RT has a higher risk of adverse side effects than treatments to other sites since there are many important organs located near the planning target volume (PTV). Recent advances in RT, including intensity modulated RT, which conforms the high dose to the complex shapes of the target volume and minimises dose to organs at risk (OAR), has led to improved survival rates and reduced toxicities. However, treatment related toxicities still occur, and can become a serious health risk if the dose received by organs close to the PTV exceeds certain thresholds

To minimise the dose delivered to healthy organs, H&N cancer patients are required to wear a skin-tight immobilisation mask that minimises patient motion. However, despite the restrictive nature of immobilisation masks, motion in the order of several mm of the tumour and surrounding tissue can still occur during and between treatment fractions. This motion can be caused by a change in the mask's fit due to patient weight loss between fractions, imperfections in the mask manufacturing and fitting process, tumour shrinkage, or treatment-related oedema. The current standard of care is to use PTV margins of 2-5 mm rather than motion tracking to account for motion and changes in the target volume. This margin, combined with intrafraction motion, leads to increased dose to surrounding healthy tissue, as well as decreased dose to the target.

The effectiveness of the cGAN segmentation method is evaluated by testing the hypothesis that the cGAN segmentation method improves GTV segmentation accuracy when compared to the current standard of care in which no GTV tracking is used. The data augmentation simulated realistic patient movement, which is achieved using a novel synthetic deformation method 510. The present invention provides a novel implementation of markerless tumour detection of H&N tumours in kV images.

The training dataset is generated from the planning CT by using a novel synthetic CT deformation method to deform each patient's planning CT to generate multiple CT volumes. From these multiple CT volumes, synthetic images in the form of digitally reconstructed radiographs (DRRs) are created and used to train a patient-specific cGAN to segment the GTV in the DRRs. To create the testing dataset, the planning CT volumes are again deformed by creating an additional realistic synthetic deformation. This additional deformation had different magnitudes to the deformations used to create the training data. The resultant deformed CT is then used to create a set of testing DRRs.

The cGANs are trained using DRRs, which are simulated 2D fluoroscopy X-ray images created from a 3D CT volume. Using a known projection geometry, DRRs can be created at different projection angles to simulate kV images acquired during RT. There are known differences between the noise properties and the image quality of kV images and DRRs, however using DRRs to train the patient-specific cGANs enables the networks to be trained without needing any additional images to be acquired. The use of DRRs for testing enables the exact location of the ground truth GTV segmentations to be known in each projection and is a useful first step in evaluating the feasibility of the cGAN segmentation method.

Database and Patient Selection. The data involved 15 patients with head and neck squamous cell carcinoma (HNSCC) and were acquired from the HNSCC database on The Cancer Imaging Archives (TCIA). For each patient, the original data consisted of a planning CT and corresponding structure file, which contained the contoured primary GTV. The 15 patients were sequentially selected based on tumour location to ensure a range of primary tumour locations and to investigate the feasibility of the method described herein. The locations of the tumour were the oropharynx (n=5), the larynx (n=5) and the nasopharynx (n=5). Patients with different tumour locations in the head and neck were selected to test the robustness of the patient-specific segmentation method.

Synthetic CT Deformations. Previous implementations of deep learning networks for markerless tumour detection and segmentation in kV images trained the network using data from 4DCT scans. The advantage of using the 4DCT for training is that the network is trained on images showing how the tumour and surrounding tissue move and deform. H&N cancer treatment planning is typically done on a regular CT scan, which presents a challenge for training a patient-specific segmentation model because regular CT scans contain a single volume whereas 4DCTs contain multiple volumes. This reduces the training dataset and results in the network being less effective at detecting and segmenting the tumour when motion occurs.

To compensate for the lack of available motion data, a CT deformation-based data augmentation method is provided that can be used to generate synthetic images depicting and reflecting realistic head motion by patients. This data augmentation method enables each patient's cGAN 100 to be trained on a patient-specific dataset containing images of H&N motion without requiring additional CT scans. Two types of movements are assumed to be the primary sources of tumour motion during RT treatment: head rotation and internal tumour motion.

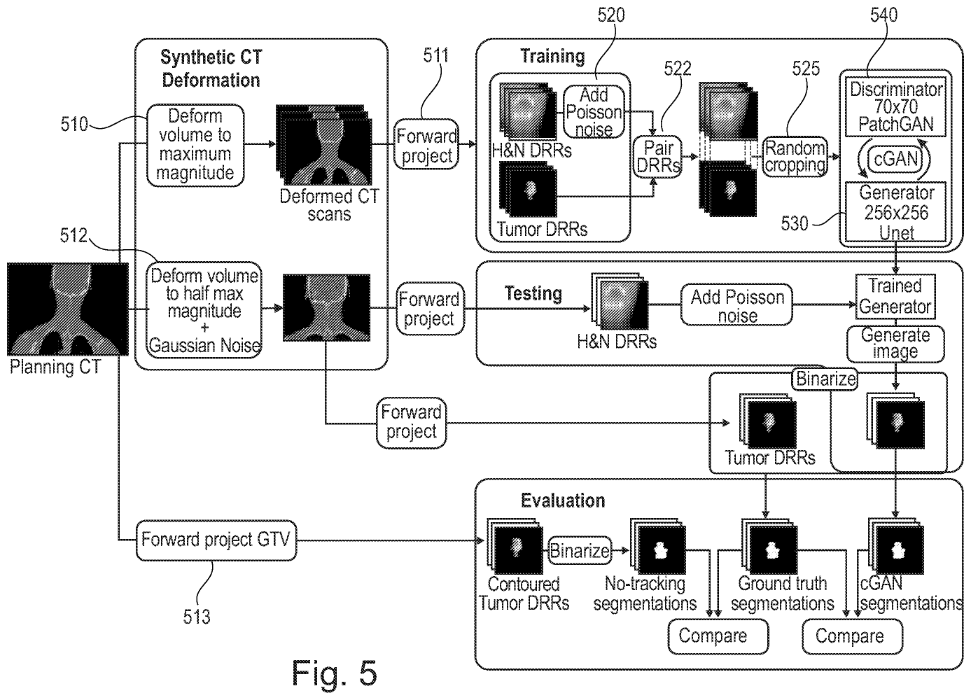

FIG. 5 shows the overall workflow for each patient specific cGAN training and accuracy evaluation. First, the planning CT and contoured Gross Tumour Volume (GTV) is deformed 510 multiple times and forward projected 511 to create the training data. Second, the same planning CT is deformed 512 at half the magnitude of the training data to create the testing data. Third, the original contoured GTV is also forward projected 513 to create the no-tracking segmentations, which assume no motion occurred in the testing data.

Using a paired 1-tail Mann-Whitney U test, the cGAN segmentation method significantly reduced the absolute segmentation centroid error when compared to the no-tracking segmentations (p<0.001). Each patient-specific cGAN network took an average of 3 hours to train.

The table below depicts the centroid error, DICE Similarity Coefficient (DSC) and Mean Surface Distance (MSD) values for the predicted cGAN segmentations. All values are mean±standard deviation.

| | | | | | |
|---|---|---|---|---|---|
| | | | Absolute | | |
| | | | centroid | | |
| | Centroid | Centroid | error | | |
| Patient | error - | error - | magnitude | | MSD |
| Number | (mm) | (mm) | (mm) | DSC | (mm) |

Oropharynx

| | | | | | |
|---|---|---|---|---|---|
| 1 | 0.7 ± 0.7 | 0.1 ± 0.9 | 1.2 ± 0.5 | 0.90 ± 0.01 | 1.5 ± 0.2 |
| 2 | −0.3 ± 0.6* | 0.2 ± 0.6 | 0.9 ± 0.4* | 0.92 ± 0.01 | 1.1 ± 0.2* |
| 3 | −0.8 ± 0.8 | −0.5 ± 0.7 | 1.2 ± 0.5 | 0.92 ± 0.01 | 1.6 ± 0.2 |
| 4 | 0.7 ± 0.5 | −0.2 ± 0.7 | 1.0 ± 0.5 | 0.89 ± 0.03 | 1.6 ± 0.4 |
| 5 | 0.7 ± 0.7 | −0.1 ± 0.7* | 1.0 ± 0.5 | 0.92 ± 0.01 | 1.9 ± 0.3 |
| All | 0.2 ± 0.9 | −0.1 ± 0.7 | 1.1 ± 0.5 | 0.91 ± 0.02 | 1.5 ± 0.4 |
| Oropharynx | | | | | |

Larynx

| | | | | | |
|---|---|---|---|---|---|
| 6 | −0.6 ± 0.5 | 0.1 ± 0.8 | 1.0 ± 0.5 | 0.90 ± 0.02 | 1.3 ± 0.3* |
| 7 | 0.7 ± 0.6 | 0.2 ± 0.6 | 1.0 ± 0.5 | 0.87 ± 0.02 | 1.6 ± 0.3 |
| 8 | −0.2 ± 0.9* | 0.0 ± 0.6* | 1.0 ± 0.6 | 0.92 ± 0.02* | 1.3 ± 0.3* |
| 9 | −0.7 ± 1.0 | 0.0 ± 0.6* | 1.2 ± 0.6 | 0.86 ± 0.03 | 1.5 ± 0.3 |
| 10 | −0.3 ± 0.4 | 0.1 ± 0.6 | 0.7 ± 0.4* | 0.91 ± 0.02 | 1.3 ± 0.3* |
| All Larynx | −0.2 ± 0.9 | 0.1 ± 0.7 | 1.0 ± 0.5 | 0.89 ± 0.03 | 1.4 ± 0.3 |

Nasopharynx

| | | | | | |
|---|---|---|---|---|---|
| 11 | 0.9 ± 0.8 | 0.2 ± 1.5 | 1.7 ± 0.9 | 0.91 ± 0.02 | 2.3 ± 0.5 |
| 12 | −0.3 ± 0.5* | 0.0 ± 0.9 | 0.9 ± 0.5 | 0.90 ± 0.02 | 2.1 ± 0.4 |
| 13 | 0.9 ± 1.0 | −0.2 ± 1.1 | 1.4 ± 1.0 | 0.92 ± 0.02 | 2.0 ± 0.6 |
| 14 | −0.4 ± 0.5 | 0.0 ± 0.5* | 0.7 ± 0.4* | 0.94 ± 0.01* | 1.4 ± 0.3* |
| 15 | 0.1 ± 0.6 | −0.3 ± 2.3 | 1.3 ± 2.0 | 0.89 ± 0.06 | 1.4 ± 0.3 |
| All | 0.3 ± 0.9 | −0.1 ± 1.4 | 1.2 ± 1.2 | 0.91 ± 0.04 | 1.8 ± 0.7 |
| Nasopharynx | | | | | |

Total

| | | | | | |
|---|---|---|---|---|---|
| All H&N | 0.1 ± 0.9 | −0.0 ± 1.0 | 1.1 ± 0.8 | 0.90 ± 0.03 | 1.6 ± 0.5 |
| patients | | | | | |

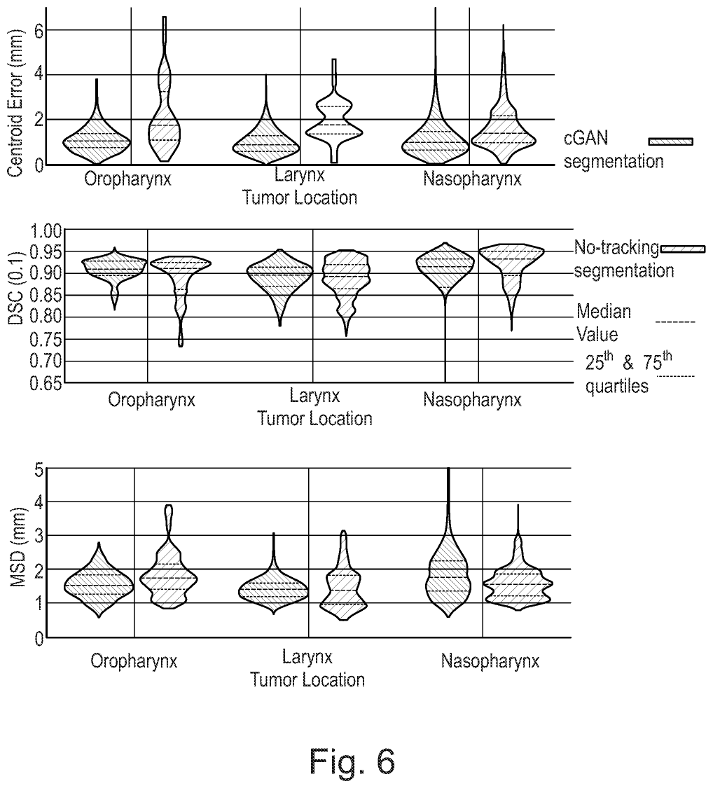

For all patients the mean±standard deviation DSC and MSD values for the cGAN segmentation were 0.90±0.03 and 1.6±0.5 mm respectively, with the 95^{th} percentile error for the DSC and MSD being [0.85, 0.94] and [0.9, 2.5] mm respectively. The distribution of both the DSC and MSD values for the cGAN segmentation method are shown in comparison to the no-tracking segmentations in FIG. 4. Using a paired 1-tail Mann-Whitney U test, the cGAN segmentation significantly reduced the MSD (p=0.031) when compared to the no-tracking segmentations. The cGAN segmentation method did not significantly improve the DSC when compared to the no-tracking method (p=0.294221) and only significantly improved the DSC for the Oropharynx tumours (p<0.0001) but not for the Larynx (p=0.203) and reduced the DSC for the Nasopharyx (p<0.0001). The violin plots in FIG. 6 shows the overall distributions of the cGAN performance across centroid, MSD and DSC metrics.

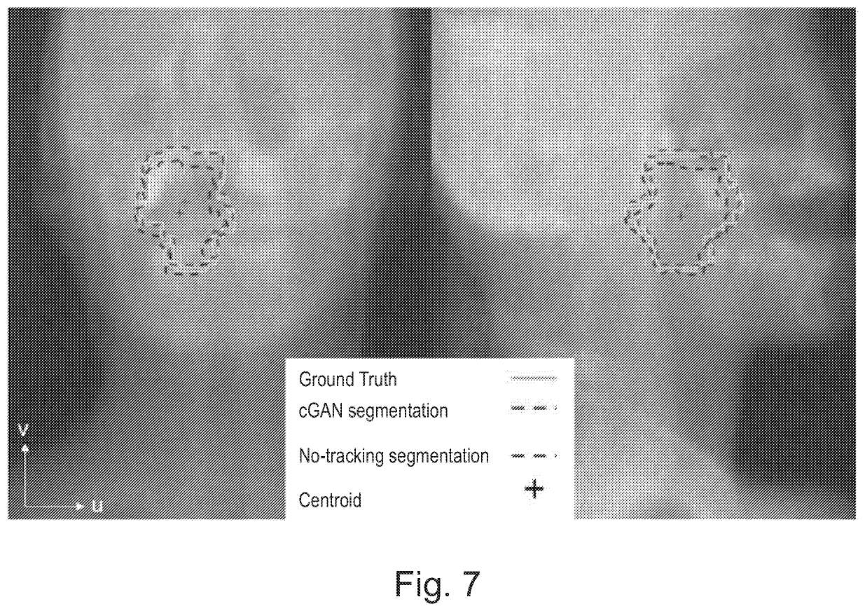

FIG. 7 demonstrates an example of the typical accuracy of the cGAN segmentations achieved by this method for a single H&N cancer patient.

### Example—Lung Tumours and Heart Tracking

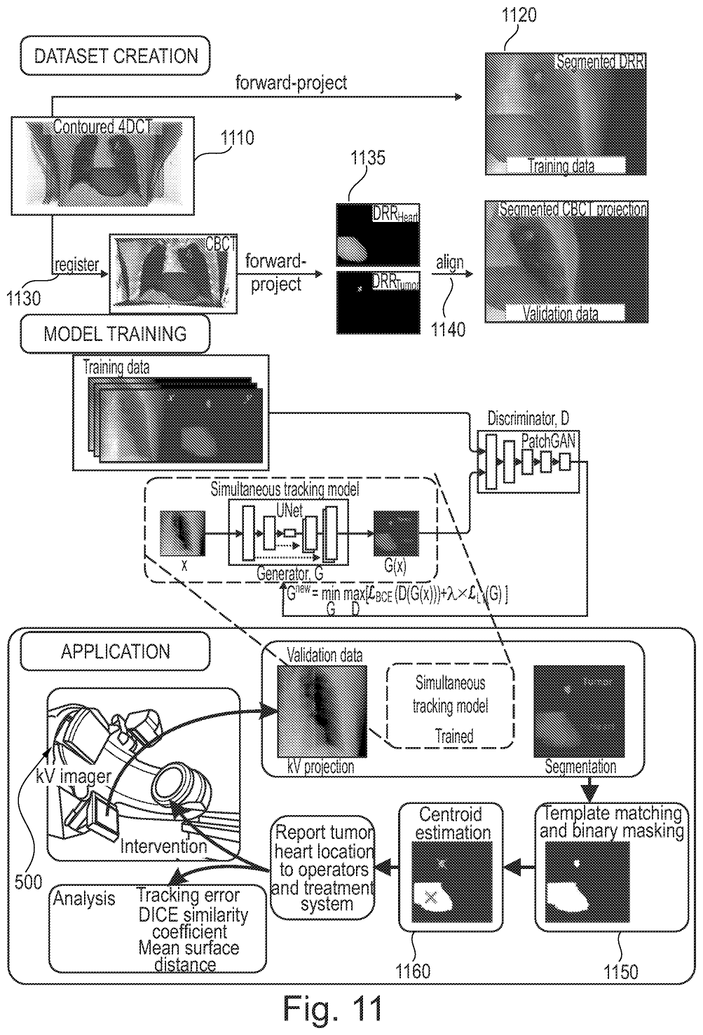

Referring to FIG. 11, prior to the commencement of treatment, as part of the conventional clinical workflow for lung SABR a 4DCT is acquired and contoured 1110 to plan the radiation treatment delivery. Stereotactic ablative body radiation therapy (SABR) has become the standard of care in patients with peripherally located stage I-IIA non-small cell lung cancer who are medically inoperable or refuse surgery. Prior to treatment a dataset of digitally reconstructed radiographs (DRRs) is generated 1120 based on the treatment planning 4DCT and used to train a patient-specific deep-learning model. During treatment, Simultaneous Tumor and OAR Tracking (STOART) is deployed to segment kV projections acquired during treatment, which it receives from the standard-equipped on-board kV imager 500 of the treatment system.

A Pix2Pix conditional Generative Adversarial Network (cGAN) for image-to-image translation tasks is tuned for robust, unsupervised learning of 16-bit greyscale kV projections. The generator network G (U-Net) is configured to learn how to translate an input kV projection into the DRRs of multiple structures (segmentation DRRs), while being supervised by a discriminator network D (70×70 PatchGAN). Discriminator network D is configured to classify whether the output segmentation DRR is similar to a ground truth, thus guiding the generator network G to continuously improve its ability to segment the input kV projections. The segmentation DRR is then binarised to generate a binary mask 1150 for each structure.

The details of the used dataset are stated below. Each segmented structure is allocated a separate image channel of the segmentation image. The training images were resized to 525×525×3 pixels (length×height×channel) and then randomly cropped to a size of 512×512×3 pixels (+2.5 mm) for augmentation each time before they were loaded into the network. The testing images were resized to 512×512×3 pixels directly before entering the network. Next, each channel of the image is normalised separately between 0 and 1 by subtracting the minimum pixel value and then dividing by the maximum pixel value. A stable network convergence is achieved through a learning rate of 0.001, an exponential learning rate decay and the shared loss function L_{BCE}. The models were trained for four epochs/144,000 iterations on a computer with an AMD Ryzen® 9 3950X 16-core central processing unit (CPU), 64 Gb RAM as memory and an NIVIDIA® RTX2080TI® Graphics Processing Unit (GPU).

For testing, the trained G is used to segment the kV projection. The segmentation image channels were separated to receive individual segmentation images for the tumour and the heart. Specifically for the tumour, the appearance of the segmentation is regularised by template-matching the forward-projected tumour contour from the end-exhale 4DCT (DRR_{Tumour}) using normalised cross-correlation. Next, label maps were created for both segmentations by separating the segmentation from the background and the 2D centroid of each label is determined 1160.

The patient data originated from in total seven lung cancer patients consisting of five lung SABR patients taken from the clinical trial LightSABR and two lung cancer patient with centrally located tumour from the publicly available VCU dataset. The patients were selected for their appropriate anatomy such that the tumour is fully visible, and the heart is at least partly visible in the kV projections. The dataset per patient consisted of in total 36,000 DRRs generated from 4DCTs for training and 250-700 kV projections from one to three cone-beam CT (CBCT) scans for testing.

The original dataset consisted of ten-phase 4DCTs with tumour contours for all ten phases and the heart contour in the end-exhale phase. Firstly, the heart contour is propagated to all 4DCT phases using deformable image registration (DIR) 1130 with Plastimatch. Next, segmented structure volumes 1135 were created for the tumour (4DCT Tumour) and the heart (4DCT_{Heart}) by replacing the voxel values outside the respective contour in each 4DCT phase with air density (−1000 Hounsfield Units). The volumes were translated such that the DRR and kV projections were rigidly aligned 1140, because the pre-treatment CBCT scans were acquired before alignment with the treatment plan.

The process of creating the training and testing images is illustrated in FIG. 11. For training, DRRs were created by forward-projecting each of the ten phases of the 4DCT (DRR_{4DCT}), 4DCT_{Tumour} (DRR_{Tumour}) and 4DCT_{Heart} (DRR_{Heart}) equidistantly over a 360 degree imaging arc with a spacing of 0.1 degrees using RTK and the CBCT scan geometry (SDD=1500 mm, detector offset of 148 mm, field of view of 298×397 mm², 1024×768 pixels with a size of 0.39×0.39 mm², 120 kVp). The DRRs were stacked to create a multi-channel image with a size of 2048×768×3 pixels (length×height×channel) as follows. The left half of the training image represented the input image (kV projection), where all three image channels were filled with the DRR_{4DCT}. The right side of the training image represented the ground truth segmentation and is created by filling two image channels with the DRR_{Tumour} and one channel with the DRR_{Heart}.

For testing, the kV projections were CBCT projections of free-breathing patients acquired over a 200-degree imaging arc at the start of the lung SABR treatment. The ground truth segmentations in the kV projections were determined manually. Firstly, the tumour and heart contour were propagated from the end-exhale 4DCT phase to the CBCT of each treatment fraction using DIR (Plastimatch). Next, the new contours were forward-projected using the imaging geometry of the kV projections. In each kV projection, fiducial markers' locations were used to guide the rigid alignment of the ground truth tumour position, while the ground truth heart position is aligned through visual inspection. Projections were discarded from the dataset if the poor soft tissue contrast made it impossible for the operator to label the ground truth heart position. The surrogacy uncertainty (SU) of the markers to define the ground truth tumour position is previously measured for the LightSABR dataset. It is given as the 95% confidence interval of the differential motion between the surrogate and the target across the ten 4DCT phases. The SU in the LR/AP direction in the kV projection is the mean of the individual SUs in LR and AP directions.

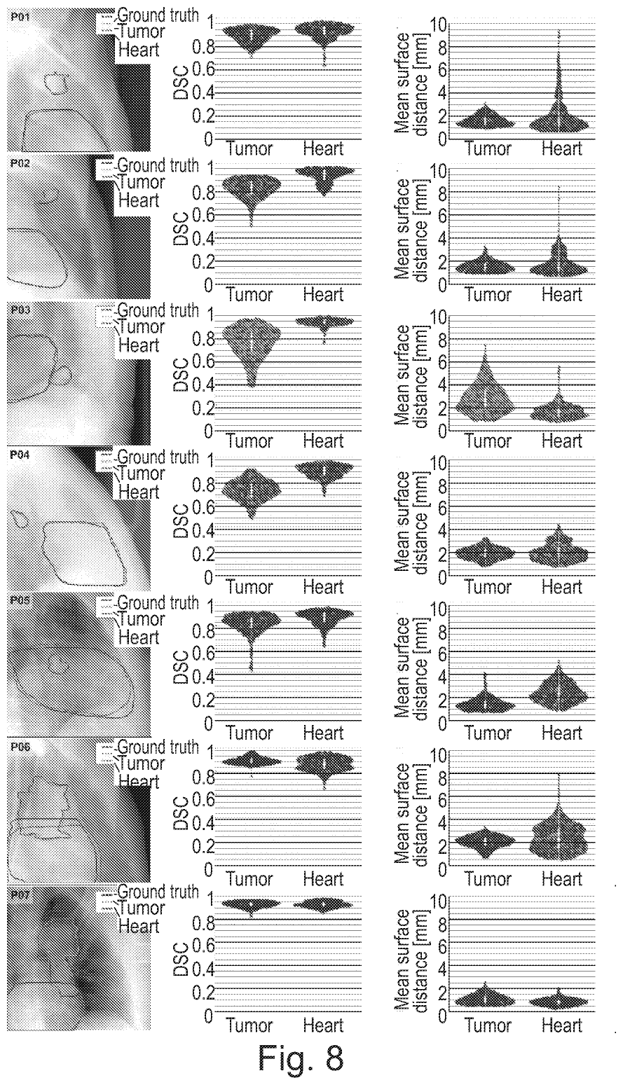

Referring to FIG. 8, STOART is applied to simultaneously segment the lung tumour and the heart in kV projections from in total 17 treatment fractions of seven lung cancer radiotherapy patients. The tracking accuracy is determined as the mean difference (±standard deviation) between the centroids of the labels in the segmentation and their respective ground truth. The 2D segmentation similarity is determined by the Dice-similarity-coefficient (DSC) and the mean surface distance (MSD). Where the heart volume is only partly visible, only the visible section is compared. The computation time is measured as the time between the kV projection entering the deep-learning model and the output of the segmentation.

From a visual inspection of the algorithm performance, STOART performed better for the anterior-posterior projections where there is higher soft-tissue contrast. STOART performed worse for the projection angles where the segmented structures are overshadowed by structures of high-contrast, such as the spine. A direct correlation is found between the tracking accuracy for individual patients and their respective surrogacy uncertainty. It is found that the largest tracking errors occurred when the target motion is outside the learned motion range of the 4DCT.

A patient-specific deep-learning model 100 for simultaneous segmentation of the tumour and heart in kV projections for motion management in real-time during radiotherapy on a conventional radiotherapy treatment system is feasible. The tracking accuracy and precision as well as the MSD over all seven patient cases were <2.0 mm for both the tumour and the heart. The mean overlap of the segmentations and the ground truth measured by the DSC is 0.82±0.08 for the tumour and 0.89±0.06 for the heart. The individual results of tracking tumour and heart compare well to other work on X-ray based marker-less tracking of a single tumour and single cardiac structures, although the latter has not yet been investigated for X-ray based tracking on patients.

STOART is a framework that is capable of simultaneously tracking two (and potentially more) targets independently and overcome the influence of intra- and inter-fractional changes in anatomy. The heart is selected as the primary OAR for this embodiment as it is the most visible OAR on KV images and therefore most suitable for determining feasibility. Compared to the current state-of-the-art, having multiple models for multiple targets, STOART can potentially overcome challenges of computational complexity, usability, validation, and maintenance for better applicability in the clinic. As simultaneous tumour and OAR tracking is a fast (computation time <50 ms per image) software solution, it could be deployed into a real-time image guided radiation therapy workflow on a conventional linear accelerator. Hence, STOART can potentially widen the therapeutic window of radiotherapy for tumours in close proximity to OARs. This technique, particularly the heart tracking as implemented on a conventional radiotherapy accelerator, may also have applicability to other novel treatment techniques and potentially support the feasibility, efficacy, and safety of SABR to the myocardial scar for patients with refractory ventricular arrhythmias.

Easy integrability into the clinical lung SBRT workflow is assisted by training the patient-specific cGAN model on synthetic DRRs, before it is applied to kV projections. The data domain shift between synthetic and real-world data is overcome by taking the following approximations into account: (i) The DRRs were generated to mimic the real kV-projections as closely as possible, by including image noise 520 and realistic anatomic motion. (ii) A clinical alignment uncertainty of ±2.5 mm is included in the data augmentation process. (iii) The segmentation for the tumour is regularised by template-matching the treatment planning tumour shape to the model output. The latter is found to improve STOART robustness against overfitting by the cGAN model and is built on the assumption that the tumour shape does not change between treatment planning and delivery. A direct correlation is found between the tracking accuracy for individual patients and their respective surrogacy uncertainty.

There is an overall symmetric distribution of tracking errors, with occasional outliers. In a clinical implementation, these occasional outliers could lead to false positive target tracking results. However, since they occur randomly, they could potentially be smoothed out using e.g. a Kalman filter or can potentially be selected based on the score measured by the discriminator network. However, this will be part of future work. Firstly, only seven patients with a total of 17 treatment fractions could be included in this embodiment, because of the requirements of both the tumour and the heart being visible in kV projections. Therefore, the results may only be an approximation for STOART performance on a large patient cohort. A major difficulty for the evaluation of tracking methods in general is the lack of ground truth information about the target position in the kV projection. Fiducial markers are subject to surrogacy errors and may therefore limit the accuracy of the ground truth tumour segmentation. The tumour tracking accuracy is of similar size as the uncertainty of the ground truth tumour position as previously reported. Secondly, this embodiment investigated STOART performance in segmenting CBCT projections, however for the purpose of intrafraction image guidance, intrafraction fluoroscopic kV projections need to be analysed. The latter have a lower signal to noise ratio because of the scattered radiation from the MV treatment beam and are therefore more challenging to segment compared to CBCT projections. Alternatively, techniques to reduce the influence of the MV scatter in kV projections e.g., pausing the MV beam during kV projection acquisition, can be implemented. Lastly, the fiducial markers appear as high-contrast features in the DRRs and kV projections, which may potentially bias the tumour segmentation. However, the fiducial markers were not implanted inside the tumour volume and therefore also not included in the segmentation DRR of the network output.

Using STOART for tracking multiple structures in kV projections in a simulated clinical environment is feasible. There are several steps in order to use STOART for a guided SABR treatment delivery in a clinical trial. These steps include the implementation of a model to infer the 3D structure coordinates from the 2D kV projection, a prospective implementation as well as the development of a quality assurance procedure. STOART may enable real-time treatment adaptation to target motion for conventional clinical workflows and minimise dose to the surrounding OARs. Adapting the radiation treatment delivery to the tumour and OARs simultaneously is desirable because it may potentially widen the therapeutic window of radiotherapy for tumours in close proximity to OARs.

Although the present invention has been described in detail in relation to examples for the specific interventional procedure of radiation therapy, it is envisaged that the present invention may be applied to other interventional procedures where image guidance may be relied upon such as needle biopsy and minimally invasive surgery.

It is envisaged that population-based trained conditional Generative Adversarial Networks (cGANs) may be efficacious for organs exhibiting high inter-patient similarity, such as the prostate, heart, uterus, kidneys, thyroid or pancreas. Specifically, in another embodiment of the present invention, a population model trained on Direct Radiograph Renderings (DRRs) derived from a substantial number of patients of a particular demographic or type could be successfully deployed for new patients which may provide efficiency by eliminating or reducing the need for patient-specific individually trained cGAN models. However, the benefits of a population-based cGAN model diminishes when the target organ exhibits substantial intra-population variation, rendering the population model inadequate for accurate tracking in such circumstances.

It is envisaged that the present invention can be applied to biopsy needle procedures. Often, when performing a biopsy, physicians must guide the needle into a specific region of interest within the body, such as a tumour or a suspicious mass, to collect tissue samples. This requires an accurate localisation of the region of interest to ensure that the needle is correctly placed. By using the system 10, the imaging of the target area can be analysed with a patient-specific, individually trained artificial neural network such as the cGAN 100 described. During treatment, the cGAN can precisely determine the position of the anatomical objects of interest, such as the tumour or mass, and consequently aid in the accurate placement of the biopsy needle. By outputting these determined positions, physicians can have a more precise guide, minimising the risk of damaging healthy tissues during the procedure and improving the accuracy of the biopsy.

It is envisaged that the present invention can be applied to non-invasive surgery procedures such as endoscopic surgery, which relies heavily on accurate imaging to guide the treatment. In endoscopic surgery, the accurate determination of the position of the anatomical objects and structures can help guide the endoscope and the surgical instruments to the exact location of the area to be treated, improving the precision of the surgical procedure. This can lead to improved treatment outcomes and reduce potential complications.

In some embodiments of the present invention, an advantage provided includes patient-specific analysis where the artificial neural network is trained on a patient-specific basis. This allows for a highly personalised analysis of the patient's anatomy, accounting for unique anatomical structures or variances. This level of customisation improves the precision of target area identification and thus, the efficacy of the treatment or medical procedure. Another advantage provided by some embodiments is real-time image guidance during an interventional procedure which facilitates immediate adjustments during the procedure, ensuring the medical device's accurate placement or movement, improving overall procedural success. Another advantage provided by some embodiments is improved precision: The ability to determine the position of one or more anatomical objects of interest in the target area accurately and accounting for body motion allows for higher precision in treatment delivery. This is particularly important in procedures like radiation therapy or biopsy, where accuracy is critical in minimising damage to surrounding healthy tissue. Another advantage provided by some embodiments is increased safety. With high precision enabled, there is a reduced risk of harm to the patient. By minimising the potential damage to non-target areas and most (if not all) treatment reaching the target area, the safety and efficacy are enhanced. Another advantage provided by some embodiments is an efficient use of sometimes scarce or sparse imaging data through re-using data already in existence from treatment planning (e.g. contour delineation) and avoiding the requirement of additional manual annotation by physicians on top of their usual clinical workflow.

In this specification, terms such as ‘processor’, ‘computer’, and so forth, unless otherwise required by the context, should be understood as referring to a range of possible implementations of devices, apparatus and systems comprising a combination of hardware and software. This includes single-processor and multi-processor devices and apparatus, including portable devices, desktop computers, and various types of server systems, including cooperating hardware and software platforms that may be co-located or distributed. Hardware may include conventional personal computer architectures, or other general-purpose hardware platforms. Software may include commercially available operating system software in combination with various application and service programs. Alternatively, computing or processing platforms may comprise custom hardware and/or software architectures. For enhanced scalability, computing and processing systems may comprise cloud computing platforms, enabling physical hardware resources to be allocated dynamically in response to service demands. While all of these variations fall within the scope of the present invention, for ease of explanation and understanding the exemplary embodiments described herein are based upon single-processor general-purpose computing platforms, commonly available operating system platforms, and/or widely available consumer products, such as desktop PCs, notebook or laptop PCs, smartphones, tablet computers, and so forth.

In particular, the term ‘processing unit’ is used in this specification (including the claims) to refer to any suitable combination of hardware and software configured to perform a particular defined task, such as generating and transmitting authentication data, receiving and processing authentication data, or receiving and validating authentication data. Such a processing unit may comprise an executable code module executing at a single location on a single processing device, or may comprise cooperating executable code modules executing in multiple locations and/or on multiple processing devices. For example, in some embodiments of the invention authentication processing may be performed entirely by code executing on a server, while in other embodiments corresponding processing may be performed cooperatively by code modules executing on the secure system and server. For example, embodiments of the invention may employ application programming interface (API) code modules, installed at the secure system, or at another third-party system, configured to operate cooperatively with code modules executing on the server in order to provide the secure system with authentication services.

Software components embodying features of the invention may be developed using any suitable programming language, development environment, or combinations of languages and development environments, as will be familiar to persons skilled in the art of software engineering. For example, suitable software may be developed using the C programming language, the Java programming language, the C++ programming language, the Go programming language, and/or a range of languages suitable for implementation of network or web-based services, such as JavaScript, HTML, PHP, ASP, JSP, Ruby, Python, and so forth. These examples are not intended to be limiting, and it will be appreciated that convenient languages or development systems may be employed, in accordance with system requirements.

In the exemplary system, the endpoint devices each comprise a processor. The processor is interfaced to, or otherwise operably associated with, a communications interface, one or more user input/output (I/O) interfaces, and local storage, which may comprise a combination of volatile and non-volatile storage. Non-volatile storage may include solid-state non-volatile memory, such as read only memory (ROM) flash memory, or the like. Volatile storage may include random access memory (RAM). The storage contains program instructions and transient data relating to the operation of the endpoint device. In some embodiments, the endpoint device may include additional peripheral interfaces, such as an interface to high-capacity non-volatile storage, such as a hard disk drive, optical drive, and so forth (not shown in FIG. 1).

The processor of a computer of control system 30 is interfaced to, or otherwise operably associated with a non-volatile memory/storage device, which may be a hard disk drive, and/or may include a solid-state non-volatile memory, such as ROM, flash memory, or the like. The processor is also interfaced to volatile storage, such as RAM, which contains program instructions and transient data relating to the operation of the server.

In a conventional configuration, the storage device maintains known program and data content relevant to the normal operation of the server. For example, the storage device may contain operating system programs and data, as well as other executable application software necessary for the intended functions of the server. The storage device also contains program instructions which, when executed by the processor, instruct the server to perform operations relating to an embodiment of the present invention, such as are described in greater detail. In operation, instructions and data held on the storage device are transferred to volatile memory for execution on demand.

The processor is also operably associated with a communications interface in a conventional manner. The communications interface facilitates access to the data communications network.

In use, the volatile storage contains a corresponding body of program instructions transferred from the storage device and configured to perform processing and other operations embodying features of the present invention.

It should be appreciated that while particular embodiments and variations of the invention have been described herein, further modifications and alternatives will be apparent to persons skilled in the relevant arts. In particular, the examples are offered by way of illustrating the principles of the invention, and to provide a number of specific methods and arrangements for putting those principles into effect.

Accordingly, the described embodiments should be understood as being provided by way of example, for the purpose of teaching the general features and principles of the invention, but should not be understood as limiting the scope of the invention, which is as defined in the appended claims.

1. An image guidance method for treatment by a medical device, comprising:
imaging a target area to which the treatment is to be delivered;
during the interventional procedure, analysing an image from the imaging with a patient-specific, individually trained artificial neural network to determine the position of at least one or more anatomical objects of interest present in the target area; and
outputting the determined position(s).

2. The method according to claim 1, wherein the artificial neural network is a conditional Generative Adversarial Network (cGAN).

3. The method according to claim 1, wherein the treatment is an interventional procedure being any one from the group consisting of: guided radiation therapy, needle biopsy and minimally invasive surgery.

4. The method according to claim 1, wherein the anatomical object of interest is any one from the group of: soft tissue and hard tissue.

5. The method according to claim 4, wherein the soft tissue is an organ or tumour.

6. The method according to claim 1, wherein the image is an X-ray image.

7. The method according to claim 3, wherein the determined position(s) is output to a radiation therapy system for the guided radiation therapy.

8. The method according to claim 7, further comprising:
identifying the target area to which radiation is to be delivered on a basis of the outputted positions.

9. The method according to claim 8, further comprising:
directing a treatment beam from the radiation therapy system based on a position of the identified target area.

10. The method according to claim 9, further comprising:
tracking the target area by reference to successive output of positions over time; and
directing the treatment beam at the target based on said tracking.

11. The method according to claim 10, wherein directing the beam based on the position of the identified target area includes adjusting or setting one or more of the following parameters of the radiation therapy system:
at least one geometrical property of said at least one emitted beam;
a position of the target relative to the beam;
a time of emission of the beam; and
an angle of emission of the beam relative to the target area about a system rotational axis.

12. An image guidance system for treatment provided by a medical device comprising:
an imaging system arranged to generate a succession of images of a target area for directing the treatment provided by the medical device;
a control system configured to:
receive images from the imaging system;
analyse the images with a patient-specific, individually trained artificial neural network during the treatment to:
determine the position of the target area; and
adjust the medical device using the determined positions to direct the treatment to the target area.

13. The system according to claim 12, wherein the artificial neural network is a conditional Generative Adversarial Network (cGAN).

13. The system according to claim 12, wherein the treatment is guided radiation therapy.

14. The system according to claim 13, wherein the medical device is a radiation therapy treatment system comprising a radiation source for emitting at least one treatment beam of radiation.

15. The system according to claim 14, wherein the treatment beam is directed to the target area.

16. A computer software product comprising a sequence of instructions storable on one or more computer-readable storage media, said instructions when executed by one or more processors, cause the processor to:
receive an image, from an imaging system, of a target area for directing treatment by a medical device;
analyse the image with a patient-specific, individually trained artificial neural network to determine the position of one or more anatomical objects of interest present in the target area; and
output the position of the one or more anatomical objects of interest to the medical device.

17. A method of monitoring movement of an organ or portion of an organ or surrogates of the organ during treatment, comprising:
directing treatment to at least a portion of an organ in a body part or human or animal subject;
imaging multiple two-dimensional images of the organ or surrogates of the organ from varying positions and angles relative to the body part;
digitally processing at least a plurality of the multiple two-dimensional images using a one or more computers with a software application executing patient-specific, individually trained artificial neural network; and
displaying estimated three-dimensional motion of the organ or portion of the organ in the body part based on output from the digital processing.

18. The method according to claim 17, wherein the multiple two-dimensional images are obtained using a linear accelerator gantry mounted kilovoltage X-ray imager system.

19. A method of training a conditional Generative Adversarial Network (cGAN) for determining the position of one or more anatomical objects of interest present in the target area of an image of a patient, the method comprising:
generating a training dataset using Direct Radiograph Rendering (DRRs) from CT images and associated contours at multiple imaging angles at high angular resolution of the patient;
training a generator network of the cGAN using the training set, where the generator network generates synthetic images of the target area;
training a discriminator network of the cGAN to evaluate smaller patches of the synthetic image generated by the generator network, where each evaluation results in a score determining whether the patch is real or fake;
calculating an averaged score of all patches evaluated by the discriminator network for each synthetic image;
adjusting the generator network based on feedback from the discriminator network to enhance the realism of generated synthetic images; and
continually optimising both the generator and discriminator networks until no further improvement can be achieved in one network without compromising the performance of the other network.

20. An image guidance method for treatment of a predetermined type of organ by a medical device, comprising:
imaging a target area to which the treatment is to be delivered;
during the interventional procedure, analysing an image from the imaging with a population-based trained conditional Generative Adversarial Network (cGAN) to determine the position of the predetermined type of organ present in the target area; and
outputting the determined position(s).

21. The method according to claim 20, wherein the predetermined type of organ is any one from the group consisting of: bones, spinal cord, prostate, heart, uterus, kidneys, thyroid and pancreas.
